SEQUENCE LISTING (2 sequences)

SEQ ID NO: 1 - DNA, 3549 bp, Pisum sativum
  P. sativum SBE1 cDNA
ccattcttcaccgtacaatacatccctatctataataaacaacaattttcttctcatcattatatcattttattcaattattaaatagttttattctttcaagaagttaatgcgaatcattttttactctgtatgtcatcacacttctcttgcctccttctactcaattttttagatctcttcttctttgcaatataaaccctaagctgatagaagaagatggtttatacaatctcaggaattcgatttcccgttcttccttctctgcacaagtcgaccttacgctgcgatcgcagagcttcatctcactctttttttctcaagaacaactcgtcatcattctctcggacctcactttatgcaaagttttcccgcgattctgaaaccaaatcttccacaattgctgaatctgataaagtacttattcctgaagatcaagataactctgtatccttggcagatcaacttgaaaatcctgatataacctcagaggatgcacagaacttggaggatttaaccatgaaagatgggaataagtacaatattgatgaatcaactagcagttatagagaggttggagatgagaaaggttctgttacgtcatcatcacttgtagatgttaacaccgacactcaagccaagaagacatcagttcattcagacaagaaagtaaaagtagataaacctaagatcattcctccacccggtactgggcagaaaatatatgagattgatccacttttgcaagctcaccgtcagcatcttgatttccgttatggacaatacaaaagaattcgggaggaaattgacaaatatgaaggtggtcttgatgcattttcgcgtgggtacgaaaagtttggcttcacacgcagtgctacaggcattacttacagggagtgggcacctggagctaagtcagcagcattagttggagatttcaacaattggaatccaaatgcagatgtaatgactaaggacgcttttggtgtatgggagatcttcttgccaaacaatgccgatggttcgccaccaattcctcatggttctcgagtcaagatccacatggatactccctctgggatcaaggactcgattcctgcttggatcaaattctctgtacaggctcctggtgaaattccatacaatggaatatactatgatcccccagaggaggaaaagtatgtcttcaaacatccacagccaaaacgaccacagtcaattagaatatatgagtcacacattggaatgagtagcccggagccaaaaatcaacacatatgcgaactttagagatgatgtactacctcgcattaaaaaacttggctataatgctgtccagattatggctatccaagaacattcttattatgctagttttgggtaccatgttactaatttctttgcacctagcagtcggtttggtactccagaagatctcaagtctctgatagacagagcccatgaactaggcctgcttgttctgatggatattgtacatagccattcctcaaataatacattggatggcctgaacatgtttgatggaactgatggtcattacttccatcctggttcacggggttatcattggatgtgggattctcgcctttttaactatggaagctgggaagtactaaggtacctactttcaaatgcaagatggtggctggatgaatataagtttgatgggtttcgatttgatggcgtcacatcaatgatgtacactcatcatggactgcaggtatcttttaccggaaattacagtgagtattttggtttagcaactgatgttgaggctgtggtttacatgatgcttgttaatgatctcattcacgggctcttccctgaagctgtttcaattggtgaagatgtgagtggaatgccaacattctgccttccgacgcaagatggtgggattggctttaactaccgcttgcatatggctgttgcagacaagtggattgagcttcttaagaaacaagatgaagattggagaatgggtgatatagtccacacactgacaaacagaaggtggctggaaaaatgtgtggtttatgctgaaagtcatgaccaggccttggttggtgataagacactagcattttggttgatggacaaggatatgtatgacttcatggctttagacaggccatctactcctctaatagatcgtggtatagcattacacaaaatgattcggcttattactatgggccttggtggtgaagggtatttgaattttatggggaatgaattcggacatcctgagtggatcgattttccaaggggtgagcaacatcttcctaatggcaaaatagttcctgggaataacaacagttatgataaatgcagacgtagatttgacttgggtgatgcagattatctaagatatcatggaatgcaagaatttgatcgagctatgcagcatctagaagagagatatggtttcatgacttctgagcaccaatatatttcacggaaaaatgaaggtgacagagttataatctttgaaagagacaacctggtctttgtcttcaattttcactggaccaacagttattcagattacaaggttggctgtttaaagccagggaaatataagattgtcttggattcagatgataccttgtttggtggcttcaataggctcaatcatactgctgagtacttcacctctgaagggtggtatgatgaccggcctcgatctttcctcgtttatgcaccttctagaacagcagtggtgtatgcccttgcggatggagttgaatcagagccaattgaactgtcagatggagtagaatcagagccaattgagctgtcagttggagttgaatcagagccaattgagctgtcagttgaagaagctgaatcagagccaattgagcggtcagttgaagaagttgaatcagagacaactcaacagtcagttgaagttgaatcagagacaactcaacagtcagttgaagttgaatcagagacaactcaatagtcagttgaagttgaaccatgatgacaagtttttctggaatgaatatatcaatttcataatggcatcagtgcaatcaagtcaaaaccagccaaccaacttaaggtgattagagcaggctctgaaactgcaaggcggagcccttgtgttaacttaatctttagtgcggcagcaaaccaacgttgaagtgtttgtacccattgcttgctaggtcaattatgtatagcacgtcaagttttttgtttttacttggggtttaataggcacaagttgcgtggcgtgtgaatgtgaaacaacaaatgtactgtcctcagatcctccgtttctgtggacatataaatcctttttacccataagtgcacaaaccttaattaaaccctggaaagttttgcgggaggccttatatatgatttctcctttcagaattaaaagttgagtcatagcaatagggccgtgctgatgaaataattgttttgtacacggtgaaaatagtattgccttactataaaatataaatggtggaacctttgcctccaattgcaaaaaaaaaaaaaaaaggaattc

SEQ ID NO: 2 - protein, 922 aa, Pisum sativum
  P. sativum SBE1 amino acid
MVYTISGIRFPVLPSLHKSTLRCDRRASSHSFFLKNNSSSFSRTSLYAKFSRDSETKSSTIAESDKVLIPEDQDNSVSLADQLENPDITSEDAQNLEDLTMKDGNKYNIDESTSSYREVGDEKGSVTSSSLVDVNTDTQAKKTSVHSDKKVKVDKPKIIPPPGTGQKIYEIDPLLQAHRQHLDFRYGQYKRIREEIDKYEGGLDAFSRGYEKFGFTRSATGITYREWAPGAKSAALVGDFNNWNPNADVMTKDAFGVWEIFLPNNADGSPPIPHGSRVKIHMDTPSGIKDSIPAWIKFSVQAPGEIPYNGIYYDPPEEEKYVFKHPQPKRPQSIRIYESHIGMSSPEPKINTYANFRDDVLPRIKKLGYNAVQIMAIQEHSYYASFGYHVTNFFAPSSRFGTPEDLKSLIDRAHELGLLVLMDIVHSHSSNNTLDGLNMFDGTDGHYFHPGSRGYHWMWDSRLFNYGSWEVLRYLLSNARWWLDEYKFDGFRFDGVTSMMYTHHGLQVSFTGNYSEYFGLATDVEAVVYMMLVNDLIHGLFPEAVSIGEDVSGMPTFCLPTQDGGIGFNYRLHMAVADKWIELLKKQDEDWRMGDIVHTLTNRRWLEKCVVYAESHDQALVGDKTLAFWLMDKDMYDFMALDRPSTPLIDRGIALHKMIRLITMGLGGEGYLNFMGNEFGHPEWIDFPRGEQHLPNGKIVPGNNNSYDKCRRRFDLGDADYLRYHGMQEFDRAMQHLEERYGFMTSEHQYISRKNEGDRVIIFERDNLVFVFNFHWTNSYSDYKVGCLKPGKYKIVLDSDDTLFGGFNRLNHTAEYFTSEGWYDDRPRSFLVYAPSRTAVVYALADGVESEPIELSDGVESEPIELSVGVESEPIELSVEEAESEPIERSVEEVESETTQQSVEVESETTQQSVEVESETTQ

(19) United States
(12) Patent Application Publication
(10) Pub. No.: US 2024/0191246 A1
(43) Pub. Date: Jun. 13, 2024

(54) FOODSTUFFS HAVING IMPROVED DIGESTION PROPERTIES

(71) Applicants: JOHN INNES ENTERPRISES LIMITED, Norwich (GB); QUADRAM INSTITUTE BIOSCIENCE, Norfolk (GB); IMPERIAL COLLEGE INNOVATIONS LIMITED, London (GB)

(72) Inventors: Claire DOMONEY, Norfolk (GB); Tracey Jane RAYNER, Surlingham (GB); Gary FROST, West Sussex (GB); Pete WILDE, Norfolk (GB); Aikaterini PETROPOULOU, London (GB); Cathrina Hansen EDWARDS, Dereham (GB)

(21) Appl. No.: 18/025,099

(22) PCT Filed: Sep. 8, 2021
(86) PCT No.: PCT/EP2021/074674

§ 371 Date: Mar. 7, 2023

(30) Foreign Application Priority Data
Sep. 8, 2020 (GB) 2014104.0

(51) Int. Cl. C12N 15/82; A23L 33/21; C12N 15/52; C12Q 1/6895; G01N 33/02

(52) U.S. Cl. CPC C12N 15/8213; A23L 33/21; C12N 15/52; C12N 15/8245; C12Q 1/6895; G01N 33/025

(57) ABSTRACT

The invention relates to methods for determining if a pea variety or a product derived from that pea variety has improved digestion properties. The invention also relates to methods of improving the digestion property of a pea, as well as method of making such plants.

### CROSS-REFERENCE TO RELATED APPLICATIONS

This application is a U.S. National Stage Application of PCT/EP2021/074674 filed Sep. 8, 2021, claiming the benefit of priority from GB2014104.0 filed Sep. 8, 2020, the entire disclosure of both applications is herein incorporated by reference.

### FIELD OF THE INVENTION

The invention relates to methods for determining if a pea variety or a product derived from that pea variety has improved digestion properties. The invention also relates to methods of improving the digestion property of a pea, as well as methods of making such plants; and to foodstuffs made from plants having improved digestion properties. Aspects of the invention further relate to methods for processing peas or pea products to maintain cellular and/or starch granule structure to provide or retain improved digestion properties.

### BACKGROUND OF THE INVENTION

The prevalence of non-communicable diseases such as obesity, type 2 diabetes (T2D), and coronary artery disease is rising, representing a major health and financial burden worldwide. Approximately 20% of the population in Europe and 37% in the USA show glucose intolerance (Eades et al., 2016, Hostalek, 2019). Abnormal postprandial blood glucose (PPG) is a major risk factor for T2D and associated metabolic diseases (O'Keefe and Bell, 2007).

The consumption of carbohydrate rich foods is a major determinant of PPG response (Wolever and Bolognesi, 1996) and glycaemic index (GI) is a method used to rank carbohydrate rich foods according to their impact on PPG (Jenkins et al., 1981). Increasing intake of low GI foods that reduce PPG has been proposed as a successful strategy to improve metabolic health and evidence from randomized control trials and systematic reviews shows a benefit of low GI diets on long term glycaemic control in T2D (Jenkins et al., 2002, Greenwood et al., 2013, Jenkins et al., 2008). There therefore exists a need to identify and develop foods that can reduce PPG levels.

Non-oil seed pulses, such as peas, chickpeas, beans and lentils are a good source of slowly-digestible carbohydrate, fibre and vegetable protein. There also exists a need to identify and develop plant varieties, in particular pea plant varieties that can also lower PPG levels.

The present invention addresses these needs.

### SUMMARY OF THE INVENTION

Elevated postprandial glucose (PPG) is a significant driver of non-communicable diseases globally. Carbohydrate-rich foods are a major determinant of PPG. Currently there is a limited understanding of how starch structure within a food-matrix interacts with the gut luminal environment to control PPG. We have identified that loss of function mutations in the starch branching enzyme I gene (SBE1) gene in pea lead to increased levels of resistant starch and a seed structure that is resistant to digestion. We believe that it is these two factors that influence post prandial glucose, and hence that such peas can provide improved digestion properties, particularly lower PPG levels. This finding allows pea varieties/plants to be selected and/or developed that will lower PPG levels, and thus the design of food products with this health benefit. Aspects of the invention relate to the discovery that the cellular structure, and/or the structure of starch granules, in resistant starch peas (which may also be referred to herein as rr peas) are important factors in the control of blood glucose; in embodiments therefore the invention relates to the processing of rr pea seed to maintain cellular and/or starch granule structure to retain the positive impact on glucose homeostasis.

One aspect of the invention provides a method for determining whether a pea plant variety or pea or pea product derived therefrom, has improved digestion properties, the method comprising determining whether the plant or pea has one or more loss of function mutations in the SBE1 (starch branching enzyme I) gene, wherein a plant or pea with one or more mutations in the SBE1 gene has improved digestion properties.

“Improved digestion properties” are defined more fully elsewhere in the present description, but it should be noted that such improved digestion properties may include reduced PPG levels after consumption when compared with a pea without such loss of function mutations. The improved digestion properties may be as a result of modified starch structure arising from the mutation (“resistant starch”), from a particular cellular structure or starch granule structure imparting resistance to digestion, from maintenance of such cellular or granular structure in a processed product, or from a combination of any or all of these.

A further aspect of the invention provides a method of improving the digestion properties of a pea or pea product, the method comprising introducing one or more mutations into the SBE1 gene of the pea plant, wherein the one or more mutation is a loss of function or partial loss of function mutation.

A further aspect of the invention provides a method of improving the digestion properties of a pea or pea product, the method comprising processing the seed of the pea in such a way as to enhance the relative resistant starch content. For example, the seed may be processed so as to retain a greater proportion of intact cells (eg, by rough milling).

Further provided is a method of producing a pea plant having improved digestion properties, the method comprising introducing one or more mutations into the SBE1 gene, wherein the one or more mutation is a loss of function or partial loss of function mutation.

Yet further provided is a method for identifying and selecting a pea plant or pea plant variety that has improved digestion properties, the method comprising detecting in the plant genome or germplasm at least one polymorphism in the SBE1 gene, wherein the polymorphism leads to a loss of function or a partial loss of function. Preferably the method further comprises introgressing the chromosomal region containing at least one polymorphism in the SBE1 gene into a second plant or plant germplasm to produce an introgressed plant or plant germplasm.

Also provided is a method of producing a food composition or nutritional supplement, the method comprising identifying and selecting a pea plant or plant variety as described herein, and producing the food composition or nutritional supplement from the plant, pea or part thereof, wherein preferably the part thereof is a seed.

In these and other aspects of the invention, the digestion property may be a reduced increase (or a reduction) in postprandial glucose (PPG) level following consumption compared to the PPG level of a pea variety, pea or product therefrom consumed without the mutation in the SBE1 gene. The SBE1 gene may encode a polypeptide as defined in SEQ ID NO: 2 or a functional variant thereof. The SBE1 gene may comprise a nucleic acid sequence comprising or consisting of SEQ ID NO: 2 or a functional variant thereof. The mutation may be a deletion, substitution or addition of one or more nucleotides. In some embodiments, the mutation may be introduced using targeted gene editing.

Also provided is a genetically altered plant, part thereof or plant cell, wherein the plant comprises a loss of function mutation in the SBE1 gene, wherein preferably the plant is a pea. Further provided is a seed obtained or obtainable from the genetically altered plant.

The invention further provides a method of increasing the level of resistant starch in a pea, the method comprising introducing a loss of function mutation in the SBE1 gene; and yet further provides a pea plant, part thereof or plant seed obtained or obtainable by said method.

Yet further provided is a food or feed composition derived from the pea plant described herein.

A further aspect of the invention provides a method for determining whether a food composition, food ingredient, or food product has improved digestion properties, the method comprising determining whether the food composition, food ingredient, or food product is prepared from a pea plant variety, pea or pea plant product having one or more loss-of-function mutations in at least one SBE1 gene; and/or the method comprising determining whether the food composition, food ingredient, or food product comprises resistant starch of the type expressed in a pea plant variety, pea or pea plant product having one or more loss-of-function mutations in at least one SBE1 gene.

### DESCRIPTION OF THE FIGURES

The invention is further described in the following non-limiting figures:

FIG. 1. Starch biosynthetic pathway in pea seeds. The contribution of different enzymes to steps in the cytosol and within the plastid and starch granule of the wild-type line, BC1/19RR, are shown in green with the metabolites in blue. The genotype of BC1/19rr carries a naturally occurring insertion in a starch-branching enzyme gene (sbel-ins, indicated by the red box, and used in this study) (Rayner et al., 2017). The mutation affects the activity of the enzyme within the structure granule of BC1/19rr, where the pathway to amylopectin is disrupted (indicated by red bar on the arrow) and starch structure is changed. Adapted from (Wang et al., 1998).

FIG. 2. Effects of acute consumption of 50 g dry weight RR and rr pea seeds and flour. t½ was determined from the modelled [13C] data to describe gastric emptying rates. t½ was defined as the timepoint at which 50% of exhaled 13CO2 is recovered. (A) Gastric emptying rates for RR and rr pea seeds group during a mixed meal experiment (trial1) (n=10). (B) Gastric emptying rates for RR and rr flour group during a mixed meal experiment (trial 1) (n=10). (C, D,) Concentration of plasma glucose for RR and rr pea seeds and flour groups during a mixed meal experiment (trial 1) (n=10). (E-H) Concentration of plasma glucose, serum insulin, GLP-1 and GIP measured for 180 min for RR and rr pea seeds group (pea seeds consumed without the mixed meal, trial 2) (n=12). (I-L) Concentration of plasma glucose and corresponding serum insulin, plasma GLP-1 and GIP for RR and rr flour group (pea flour was consumed without the mixed meal, trial 2). Analysis for flour group was performed on available paired data, (n=11). Insets show the iAUC between 0 and 300/120 for the trial 1 and 180/120 min for Trial 2. The data represent mean±SEM. Repeated Measures Anova was used to analyse time course data with pea/flour and time as within-subject factors. Fisher LSD post-hoc tests were performed between timepoints when significant pea/flourₓtime interaction was found. Paired t-tests were used for iAUCs calculations and gastric emptying data analysis. Timepoints at which values differed significantly, *p<0.05, **p<0.01, ***p<0.001. Abbreviation: iAUC, incremental area under the curve

FIG. 3 Impact of genotype structure and processing on starch digestibility. Total (A) and analytically resistant starch (B) contents of uncooked (UC), cooked (C) and cooked+simulated digested (C+SD) RR and rr pea seeds and flours. Structural characteristics of starch in uncooked and cooked flour and pea seeds; helical structure (C), chain length distribution (D). Size of cooked pea seed fragments after simulated gastric and intestinal digestion (E). Micrographs of uncooked and cooked flours (F) and sections from uncooked and cooked pea seeds (G). Compression experiments (H) using hydrated/cooked RR and rr pea seeds. One-way ANOVA was used on in vitro digestion data. Paired Student's t-test was used for the compression experiments *p<0.05, **p<0.01, ***p<0.001. ****p<0.0001

FIG. 4 The effect of structure and genotype of pea seeds and flour on small intestinal environment. (A) Small intestinal postprandial glucose curves for RR and rr pea seeds along with corresponding plasma glucose, where analysis was performed on available paired data, (n=8). (B) Individual responses expressed as AUC0-120 min for small intestinal glucose for pea seeds (n=8). (C) Postprandial small intestinal glucose curves for RR and rr flour along with corresponding plasma glucose, analysis was performed on available paired data, (n=7). (D) AUC0-120 min for small intestinal glucose for flour group (n=7). (E) RM-MCCV-PLS-DA scores plots of 1D 1H-NMR gastric samples comparing volunteers at 30 min after consumption of RR vs rr pea seed groups (n=10). Model score: R2Y 0.81, Q2Y 0.29. Dots represent the metabolic profile of each volunteer from the study cohort; blue indicates RR and red indicates rr pea seed groups. (F) Fragment from the average 600 MHz 1D ¹H-NMR spectrum of the RR (blue) vs rr (red) pea seeds showing the anomeric carbon signal (5.24 (d)) of the glucose molecule. (G) RM-MCCV-PLS-DA scores plots of 1D 1H-NMR gastric samples comparing volunteers at 30 min after consumption of RR vs rr flour groups. Model score: R2Y 0.99, Q2Y 0.85. Dots represent the metabolic profile of each volunteer from the study cohort; green corresponds to RR and orange corresponds to rr flour groups. (H) Fragment from the average 600 MHz 1D 1H-NMR spectrum of the RR flour (green) vs rr flour (orange) showing the anomeric carbon signal (5.24 (d)) of the glucose molecule. (I) Diffusion of labelled α-amylase-FITC in cooked RR and rr pea seeds (green) at different timepoints. (J) Diffusion of labelled α-amylase-FITC in cooked RR and rr flour (green) at 5 and 120 min. (K) Diffusion of labelled α-amylase-FITC at 90 min in duodenal samples in RR and rr pea seeds and flour group. Data are presented as mean±SEM. Timepoints at which values differ significantly, *p<0.05, **p<0.01. Abbreviations: AUC (area under the curve), RM-MCCV-PLSD (Repeated measures-Monte Carlo cross validation-Partial-squares-discriminant analysis), AA (α-amylase), FITC (Fluorescein isothiocyanate)

FIG. 5 Using stable-isotope 13C-enriched RR and rr pea seeds and flour to understand the digestion and fermentation process further. (A) 13C plasma glucose curves for RR and rr groups after administration of 50 g dry weight 13C-enriched pea seeds along with a mixed meal test (n=9). (B) Total AUC0-480 min of exogenous 13C plasma glucose concentrations for RR and rr seeds (n=9). (C) Postprandial plasma 13C glucose responses for RR and rr flour and (D) corresponding AUC0-480 min (n=8). (E) Fractional enrichment in urinary concentrations of 13C acetate, 13C propionate and 13C butyrate after consumption of 13C-enriched RR and rr seeds (n=10) and flour (n=9). (F) Gut microbiota weighted beta-diversity plots for RR (blue) and rr (red) peas and RR (green) and rr (orange) flour, where each data-point represents the microbial community of a single sample. 13C plasma glucose and urine samples were analysed using gas chromatography-combustion isotope ratio mass spectrometry. Beta diversity analysis was performed using the UniFrac metric calculated with QIIME 1.9.0 and visualized as a 3D principal coordinates analysis plot using Emperor. Data represent mean±SEM. Repeated Measures Anova was used for testing time course data with pea/flour and time as within-subject factors. Fisher LSD post-hoc tests were performed between timepoints when significant pea/flourₓtime interaction was found. Paired t-tests were used for AUC calculations. Time points at which values differ significantly, *p<0.05, ***p<0.001. Abbreviation: AUC, total area under the curve

FIG. 6 The effect of consuming products derived from the two pea genotypes for 28 days on glucose homeostasis and gut microbiota. (A) Postprandial plasma glucose for RR and rr lines (B) and serum insulin responses: RR (C), rr (D). (E) Nonmetric multidimensional scaling (NMDS) plots for RR and rr pea interventions before and after the consumption of pea derived food products. RR visit 1; [symbol] , RR Visit 2 [symbol] , rr Visit 1 [symbol] , rr Visit 2 [symbol] . NMDS represent a snapshot of the bacterial community structure, where dots close together have similar community structure while those far apart are dissimilar. Therefore, in this figure points closely located on the plot are more similar in local community composition than are more distant points. Samples were subsampled to the lowest number of reads (3926) which resulted in at least 97% coverage for each sample (Supplementary FIG. 8). Analysis was performed on available paired data (n=22). Subsample read counts for Bifidobacterium and Bacteroides for both lines of peas (F)

FIG. 7 (Figure S1). Effect between genotypes (RR peas-flour, rr peas-flour) after acute consumption of 50 g dry weight of products on plasma glucose and insulin responses. (A, B) Plasma glucose and corresponding serum insulin between RR whole pea seeds and flour. (C, D) Plasma glucose and corresponding serum insulin between rr whole pea seeds and flour. Repeated Measures Anova model was used for testing time course data with pea seeds and flour and time as factors. LSD Fisher post-hoc tests were performed between timepoints. Paired t-tests were used for iAUCs calculations. Normality was checked using Shapiro-Wilk test. Insets show the iAUC between 0 and 180/120 min. Timepoints at which values differed significantly, *p<0.05, **p<0.01, ***p<0.001. The data presented as mean±SEM (n=11).

FIG. 8 (Figure S2). ¹H-¹³C CP/MAS NMR spectral overlay of uncooked and cooked peas of the RR genotype (A) and uncooked and cooked peas of the rr genotype (B), with ¹³C nuclear assignment and inlay of starch glucose monomer

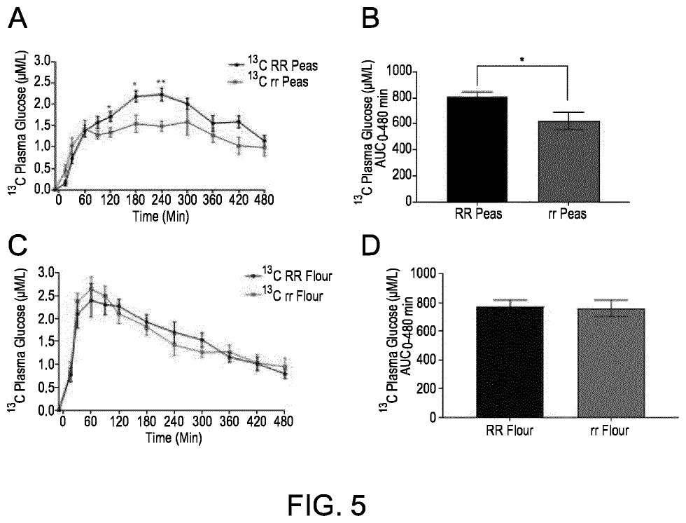

FIG. 9 (Figure S3). Light micrographs of cooked flour and peas post-simulated digestion; A) RR flour, (B) rr flour, (C) RR peas, (D) rr peas (tissue section). The rr samples showed reduced levels of gelatinisation in both the flour and the pea. All samples were stained using iodine

FIG. 10 (Figure S4). Scanning electron micrographs of uncooked and cooked whole peas, demonstrate the extent of starch gelatinization within cotyledon cells. Starch granules gelatinized more extensively in RR peas and the starch appeared to have a furry texture, possibly due to starch expanding into the surrounding protein network, and/or from amylose that had leached during cooking. In rr seeds, the protein network appeared thicker and more extensive, the cell walls appear thicker and the interstitial regions possessed different structural features, compared to RR.

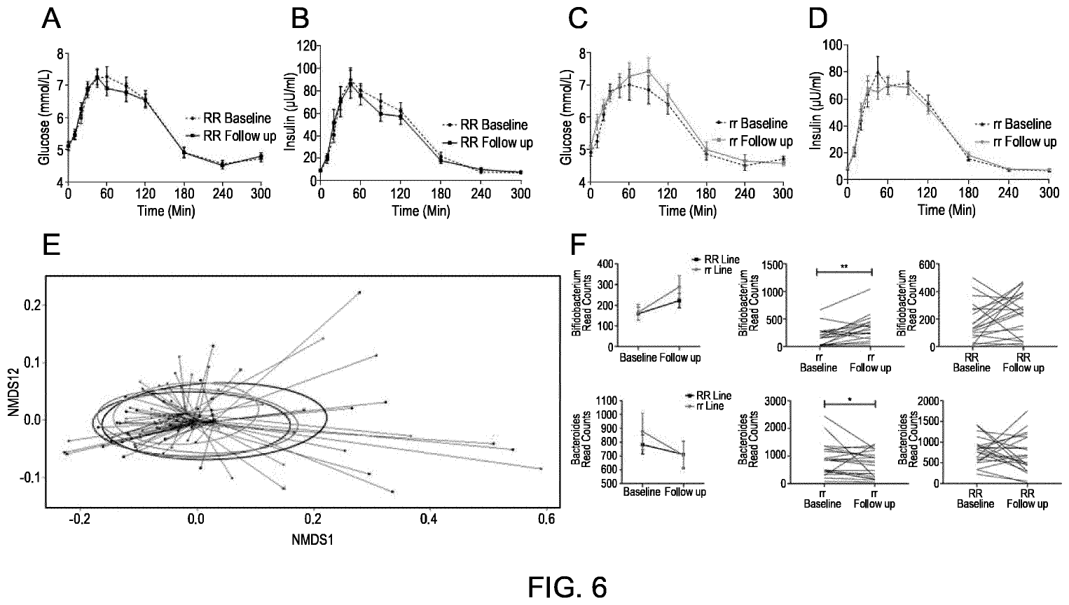

FIG. 11 (Figure S5). Distribution of the values obtained from the Intensity per nm2 and time (diffusion rate constant) of AA in RR and rr cooked pea seeds

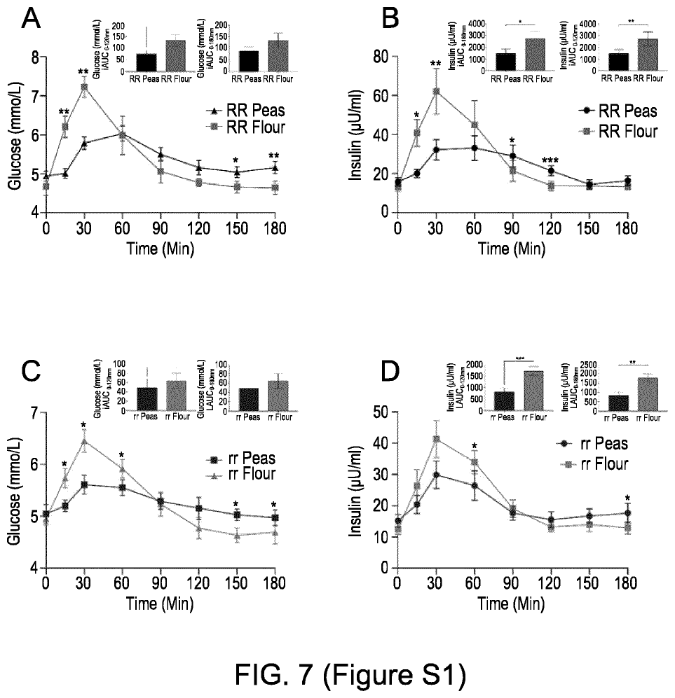

FIG. 12 (Figure S6). Consort diagram of the long-term study

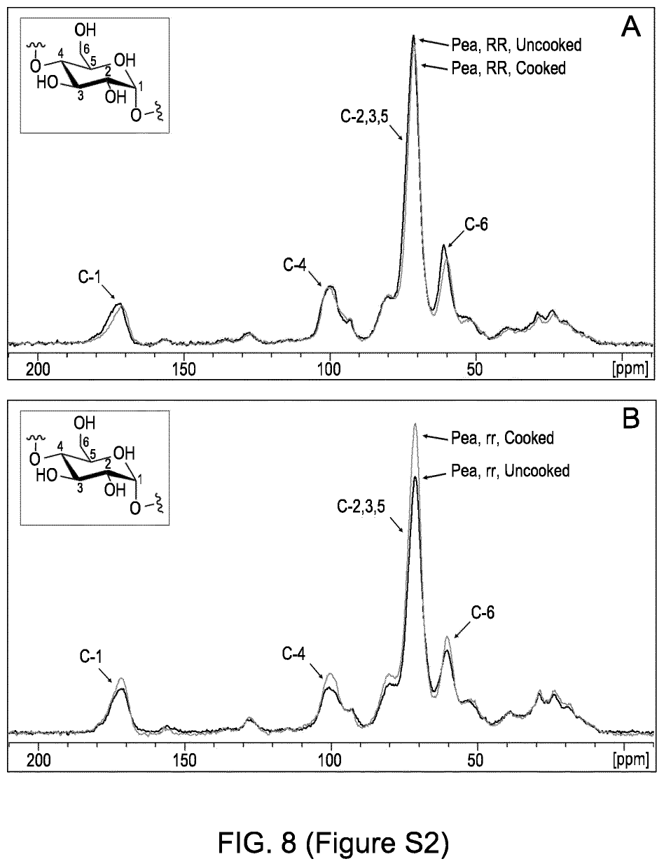

FIG. 13 (Figure S7). Rarefaction curves for all the samples used in our study. OTUs were clustered at 97% and the curves were generated using Mothur (V 1.41.3)

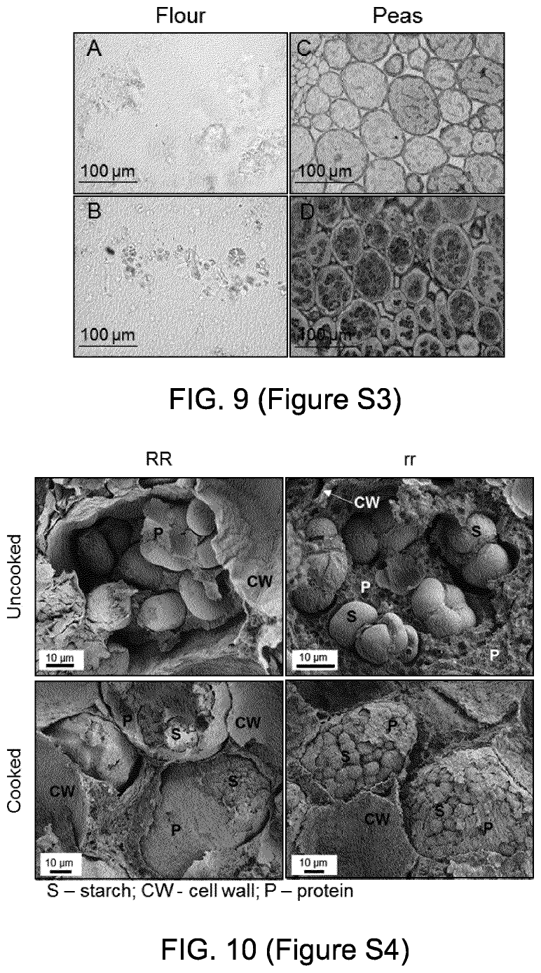

FIG. 14 (Figure S8). Trigonelline values measured at baseline and follow up visit indicating volunteer's adherence to the RR peas and rr peas supplementation.

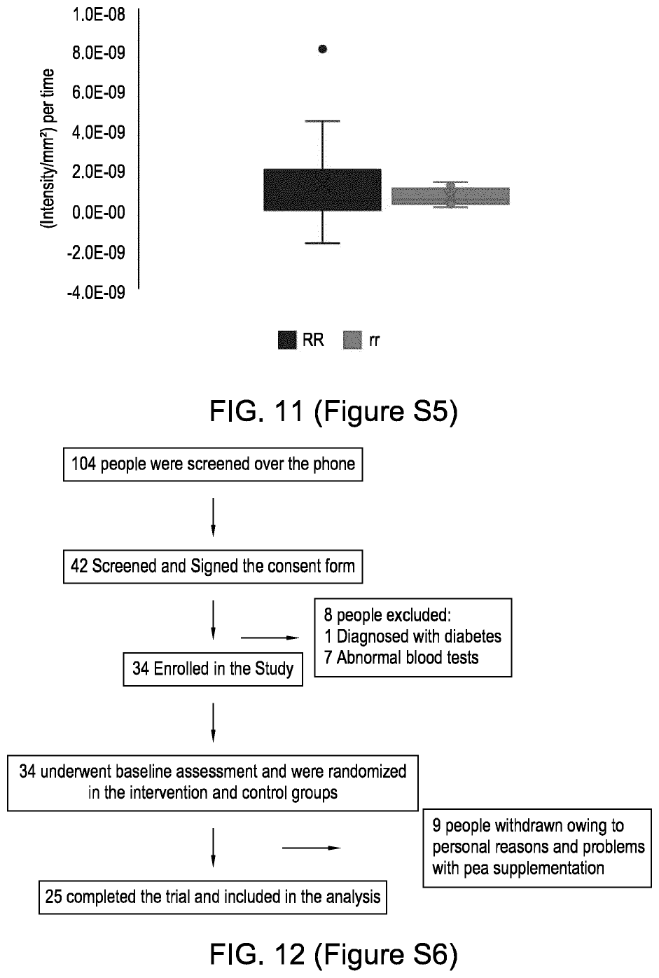

FIG. 15 (Figure S9). Preliminary crude proof-of-concept study. Glucose AUC was obtained from the area above baseline of each intervention's glucose response curve. Glucose AUC readings were 115.4±23.9, 57.2±16.1, 17.2±3.4 and 5.0±1.2 for glucose, control pea, RR and rr peas respectively. Numbers in bold indicate the glycaemic index value for each intervention. $ indicates statistical significance in rr peas, p<0.05, compared to the control peas and * indicates statistical significance in RR peas, p<0.05, compared to the control peas

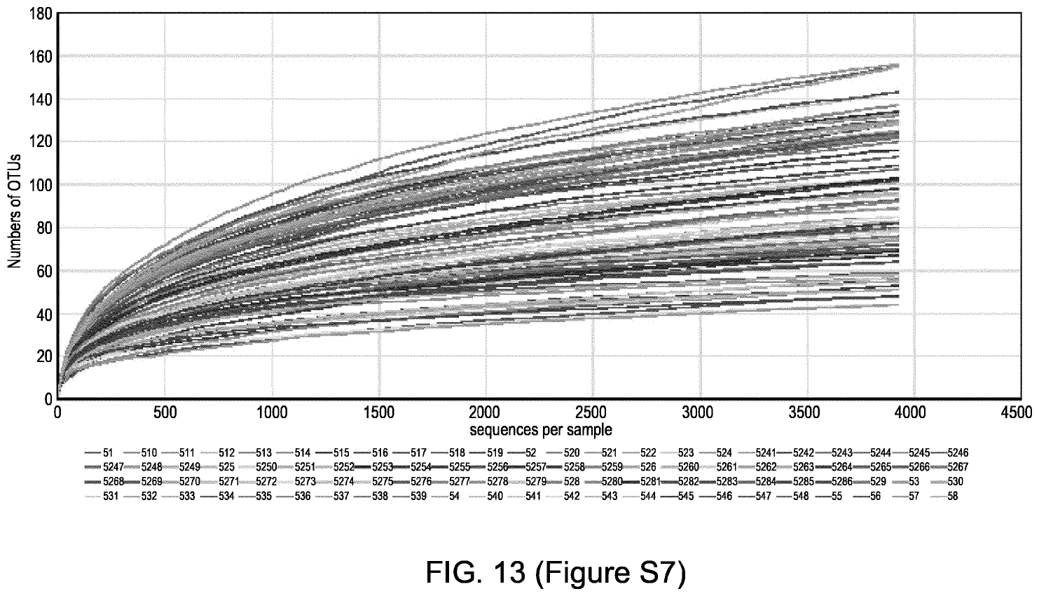

FIG. 16. Biscuit consumption. Baked goods (biscuits) were prepared from rr and RR peas, and consumed by a volunteer. The graphs show effect on glucose levels after consumption.

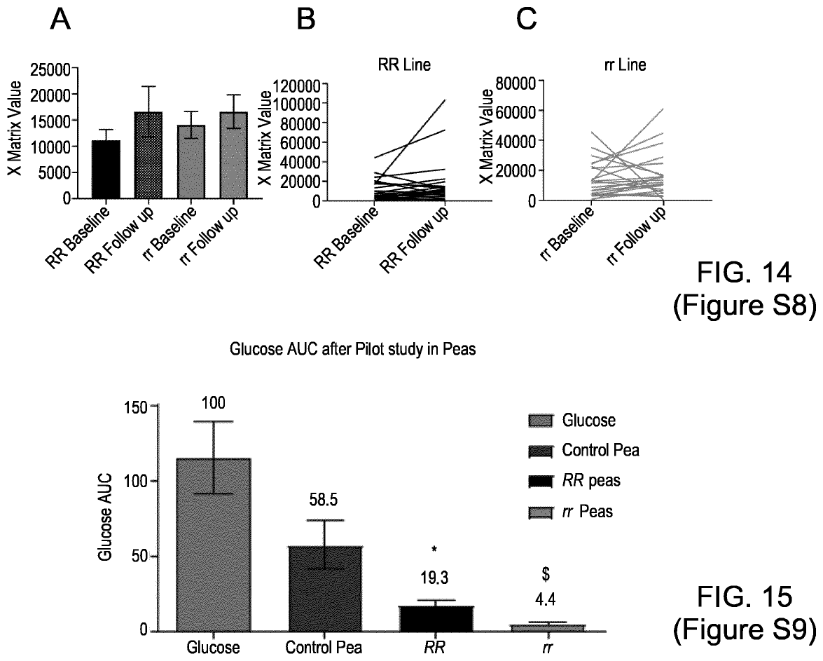

FIG. 17. Starch digestibility. Starch amylolysis assay of uncooked (above) and cooked (below) milled pea cotyledons flours from RR and rr peas and commercial varieties ‘X’, ‘Y, and ‘Z’ carrying the same mutation as rr pea. Amylolysis assay curves show release of starch amylolysis digestion products over time. Data points are mean of triplicate assays with error bars as SD, with first order curves obtained by non-linear regression to a first-order equation. Column charts show mean area under the 120 min digestibility curves obtained for each pea variety. Annotations on column charts indicate significant differences between rr and RR pea starch digestibility, results of other statistical comparisons (One-way ANOVA, Tukeys post-hoc) between commercial rr varieties are not shown, but commercial varieties XYZ were all significantly different from RR pea, and resembled digestibility of rr pea starch. All assays were performed using the same amylase-starch ratio to allow for estimation of relative differences in predicted glycaemic index as per Edwards et al 2019 Food Funct., 10, 4751-4760. These data demonstrate that in the uncooked state, rr pea starch is significantly more digestible than RR pea starch, and that once cooked, the RR pea starch is significantly more digestible than the rr pea starch. Differences in starch amylolysis profiles from rr vs RR pea starch provide a reasonable predictive indication of relative differences in postprandial glycaemic responses.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

For the purposes of the invention, a “genetically altered plant” or “mutant plant” is a plant that has been genetically altered compared to the naturally occurring wild type (WT) plant. In one embodiment, a mutant plant is a plant that has been altered compared to the naturally occurring wild type (WT) plant using a mutagenesis method, such as any of the mutagenesis methods described herein. In one embodiment, the mutagenesis method is targeted genome modification or genome editing. In one embodiment, the plant genome has been altered compared to wild type sequences using a mutagenesis method. Such plants have an altered phenotype as described herein, such as an increased level of resistant starch. Therefore, in this example increased levels of resistant starch is conferred by the presence of an altered plant genome, for example, a mutated endogenous SBE1 gene. In one embodiment, the endogenous gene is specifically targeted using targeted genome modification and the presence of a mutated gene is not conferred by the presence of transgenes expressed in the plant. In other words, the genetically altered plant can be described as transgene-free.

The aspects of the invention involve recombination DNA technology and exclude embodiments that are solely based on generating plants by traditional breeding methods.

In one aspect of the invention, there is provided a method for determining whether a pea plant variety, pea or pea plant product derived therefrom, has improved digestion properties, the method comprising determining that the plant, pea or product has one or more loss-of-function mutations in at least one SBE1 (starch branching enzyme I) gene, wherein a pea plant variety, pea or product with one or more mutations in the SBE1 gene has improved digestion properties.

A further aspect of the invention provides a method for determining whether a food composition, food ingredient, or food product has improved digestion properties, the method comprising determining whether the food composition, food ingredient, or food product is prepared from a pea plant variety, pea or pea plant product having one or more loss-of-function mutations in at least one SBE1 gene.

A yet further aspect of the invention provides a method for determining whether a food composition, food ingredient, or food product has improved digestion properties, the method comprising determining whether the food composition, food ingredient, or food product comprises resistant starch of the type expressed in a pea plant variety, pea or pea plant product having one or more loss-of-function mutations in at least one SBE1 gene.

Preferably the food composition, food ingredient, or food product comprises resistant starch making up at least 20%, 25%, 30%, 35%, 40%, 45%, 50%, 75%, 80% of the total starch content; most preferably, at least 40% of the total starch content is said resistant starch. Preferably the food composition, food ingredient, or food product is one in which digestible carbohydrates provide at least 60% of the total energy and where at least 55% of those carbohydrates is digestible starch, of which at least 40% is said resistant starch.

In another aspect of the invention, there is provided a method of improving the digestion properties of a pea or pea product, the method comprising introducing one or more mutations into the SBE1 gene, wherein the one or more mutation is a loss of function of partial loss of function mutation.

In a another aspect of the invention, there is provided a method of producing a pea plant, pea or pea product that has improved digestion properties, the method comprising introducing one or more mutations into the SBE1 gene, wherein the one or more mutation is a loss of function or partial loss of function mutation.

In a further aspect of the invention, there is a method for identifying and selecting a pea plant variety, pea or pea product that has improved digestion properties, the method comprising detecting in the plant genome or germplasm at least one polymorphism (i.e. an allele) in the SBE1 gene, wherein the polymorphism is a loss of function or a partial loss of function mutation.

In a preferred embodiment, the method further comprises introgressing the chromosomal region containing at least one polymorphism in the SBE1 gene into a second pea plant or plant germplasm to produce an introgressed plant or plant germplasm.

In a further embodiment of any of the above-described methods, the method may further comprise producing a food composition or food product, vitamin or nutritional supplement from the selected pea plant or from the pea or seed obtainable or obtained therefrom. In one embodiment, the food composition is the pea. In an alternative embodiment, the food composition may be a flour of any product derivable from a pea plant or pea or pea seed.

In one embodiment by “improving digestion properties” is meant improving at least one of the following digestion properties: plasma glucose levels, serum insulin levels, starch digestion, glycaemic response, glucose and insulin homeostasis, glucose release rates and postprandial glucose (PPG) levels in a subject once the plant, pea or product therefrom is consumed. In many situations, each of these properties may be linked, such that an improvement in one leads to an improvement in another. For example, an improvement in plasma glucose levels can result from an improvement in glucose release rates, which in turn results in improved PPG levels. In a preferred embodiment, one or more of the levels of plasma glucose, serum insulin, starch digestion, glycaemic response, glucose release rates and PPG levels in a subject are decreased following consumption of the plant, pea or pea product containing the one or more mutation in the SBE1 gene compared to consumption of a control product (this may be referred to herein as “compared to a control”); for example, consumption of a pea or pea product that does not contain a mutation in SBE1. In a most preferred embodiment, the digestion property is a reduction in PPG levels in the subject. Accordingly, in one embodiment, the method comprising determining whether consumption of the pea plant variety, pea or pea product by a subject leads to a smaller or reduced increase in PPG levels in a subject compared to the control. Alternatively, a decrease in PPG levels may be a reduction in PPG levels in a human or mammalian subject within at least 15, 30, 60, 90, 120 and 180 min of consumption of the plant, pea or product compared to a control. The decrease may be at least 25%, 50%, 75%, 80%, 85%, 90%, 95%, 100% compared to a control.

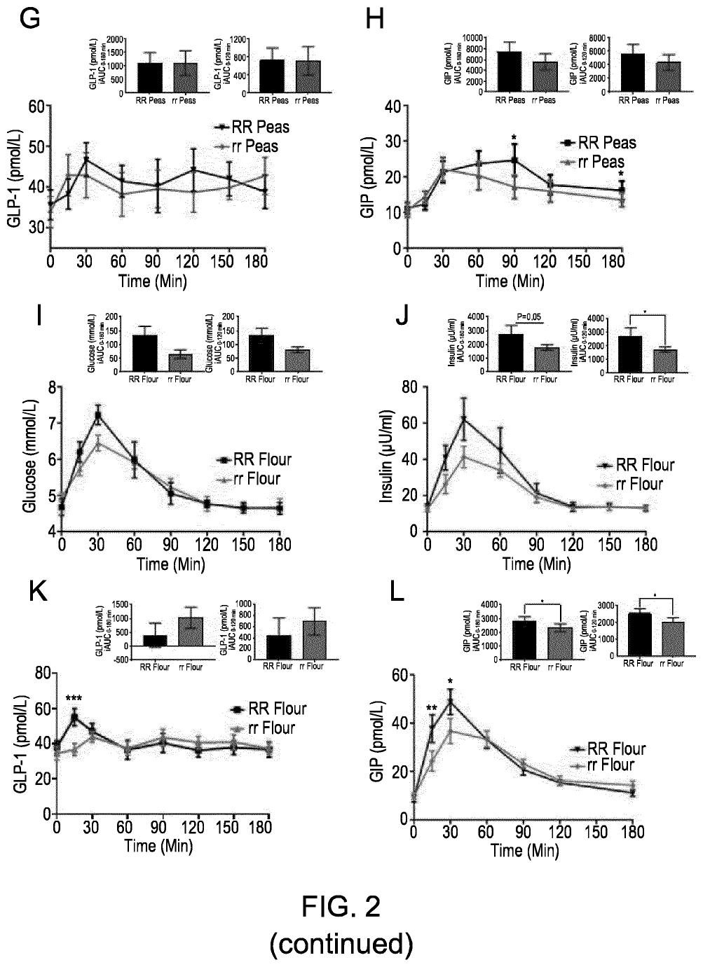

In an alternative embodiment, the digestion property is resistant starch. More preferably, levels of resistant starch are increased compared to the level of resistant starch in a control or wild-type plant, such as a plant that lacks a loss of function mutation. In one embodiment, the level of resistant starch is increased by at least 10%, 20%, 25%, 30%, 35%, 40%, 45%, 50%, 75%, 80%, 85%, 90%, 95%, 100% % or more compared to a control or wild-type plant. In preferred embodiments, an increase of at least 40% resistant starch is present. In other embodiments, the plant comprises at least 10%, 20%, 25%, 30%, 35%, 40%, 45%, 50%, 75%, 80%, 85%, 90%, 95%, 100% resistant starch as a proportion of total starch content; preferably at least 40% resistant starch. It will be appreciated that starch levels overall will vary in a plant over time, as seasons and growth stages proceed. In preferred embodiments, the resistant starch levels recited herein are either a) compared with a control plant at the same growth stage and season; or b) determined at the time that the plant is harvested (for example, where reference is made to at least 40% resistant starch as a proportion of total starch content). It will be further appreciated that effective levels of resistant starch may be increased as a consequence of such a loss of function mutation (for example, by production of forms of starch which are more resistant), and/or as a consequence of larger-scale structure in the pea seed, for example, cellular structure and/or or starch granule structure. Certain processing methods of a foodstuff may be selected to retain such larger-scale structure in a processed food; for example, processing may be selected to retain starch granule structure in a food ingredient (eg, by milling); and additional processing steps may also be selected to improve the relative amount of resistant v non-resistant starch—as will be described further herein, a combination of genotype and processing can be selected to give desirable digestion properties to a foodstuff made from the plant. In preferred embodiments of the invention, processing is selected to preserve starch granule structure in the processed food ingredient. We believe that two types of “resistant starch” can be distinguished: first, what we refer to here as Type 1 or entrapped, physically inaccessible starch; and second, “intrinsic” resistant starch. For example, Type 1 resistant starch may be found within intact cell walls which control the accessibility of digestive enzymes to the starch granules. However, we describe herein that rr peas have subcellular starch granule structures which themselves can be resistant to digestion when they form condensed or crystalline structures—that is, intrinsic resistant starch. This is associated with starches with a high amylose content or longer amylopectin chains. For the purposes of the present invention, the starch granules in the rr mutant contain a higher amylose content, and this affects their structure, size and shape (see FIGS. 3 F and G herein).

The effect of processing—and in this example, cooking—is interesting here. For the RR pea, the starch granules gelatinise during cooking in water (95° C.) making them more digestible. For the rr pea, some of the starch in the granule does not gelatinise at 95′C so is still resistant to digestion, and some of the amylose leaches out of the starch granule forming a “protective” layer around the granules (FIG. 3 G bottom right frame). We believe this behaviour reduces the digestibility of the cooked (hydrothermally processed) starch granule, making the cooked rr starch granule more resistant to digestion than the starch granules of the RR or wild type pea. Hence we see a synergy between the processing and genotype of the pea, which has not been observed before. Where we refer to “resistant starch” in the present application, primarily we refer to “intrinsic” resistant starch which is resistant by virtue of the starch granule structure.

By “pea variety” is meant a variety of pea plant from the genus Pisum, and more preferably, from the species Pisum sativum. The pea plant variety is selected from a garden pea, sugar pea and field pea. Examples of garden pea varieties include Spring, Survivor, Thomas Laxton, Wando, Garden Sweet, Mr. Big, Early Perfection, Lincoln, Little Marvel, Misty Shell, Snow Peas, Snowbird, Gray Sugar, Sugar Daddy, Oregon Sugar Pods, Mammoth Melting Sugar, Oregon Sugar Pod #2, Avalanche, Snap peas, Sugar Bon, Sugar Snap, Sugar Snappy, Super Sugar Snap VP and Sugar Ann. Accordingly, the method may comprise identifying whether one or more of these pea varieties have improved digestion properties.

By “pea” is meant the seed of a pea plant. The pea may be cooked or un-cooked. Cooked peas may refer to peas that have been roasted, boiled or steamed. Alternatively, the pea may be frozen.

By “pea plant product” is meant any product, preferably a food product, derived or derivable from a pea plant or pea or pea seed. Examples of pea products include pea flour, pea hummus, pea porridge, and mushy peas; or a product prepared from such pea products, for example, biscuits, cakes, snack foods, breads, etc, prepared from pea flour. Other pea products may include products made from or comprising whole peas; for example, fresh, frozen, or tinned peas; or products such as pea fritters, and the like. Other products made from or comprising peas include vegan or vegetarian burgers, or meat substitute products.

In some aspects of the invention, the SBE1 mutation may be in an SBE1 gene in a non-pea plant. For example, the “wrinkled” phenotype of rr peas is also observed in some non-pea seeds; these include other legumes, for example chickpea, and other crops for example maize. The invention may therefore extend to plants having a mutation in an SBE1 gene and a “wrinkled” seed phenotype similar to that seen in rr peas. It should be noted that not all “wrinkled” seed phenotypes are associated with the rr mutation—for example, mutation in the RB gene (rb) in peas results in a loss of ADP-glucose pyrophosphorylase function and produces a wrinkled seed phenotype. However, mutations at RB do not increase amylose content. Consequently rb peas (although wrinkled) do not improve digestion properties. In one aspect of the invention, there is provided a method for determining whether a non-pea plant variety, seed or plant product derived therefrom, has improved digestion properties, the method comprising determining that the plant, seed or product has one or more loss-of-function mutations in at least one SBE1 (starch branching enzyme I) gene, wherein a plant variety, seed or product with one or more mutations in the SBE1 gene has improved digestion properties.

A further aspect of the invention provides a method for determining whether a food composition, food ingredient, or food product has improved digestion properties, the method comprising determining whether the food composition, food ingredient, or food product is prepared from a non-pea plant variety, seed or plant product having one or more loss-of-function mutations in at least one SBE1 gene.

Preferably the food composition, food ingredient, or food product comprises resistant starch making up at least 10%, 15%, 20%, 25%, 30%, 35%, 40%, 45%, 50%, 75%, 80% of the total starch content; most preferably, at least 40% of the total starch content is said resistant starch. Preferably the food composition, food ingredient, or food product is one in which digestible carbohydrates provide at least 60% of the total energy and where at least 55% of those carbohydrates is digestible starch, of which at least 40% is said resistant starch.

In another aspect of the invention, there is provided a method of improving the digestion properties of a non-pea plant seed or plant product, the method comprising introducing one or more mutations into the SBE1 gene, wherein the one or more mutation is a loss of function of partial loss of function mutation.

In another aspect of the invention, there is provided a method of producing a non-pea plant seed or plant product that has improved digestion properties, the method comprising introducing one or more mutations into the SBE1 gene, wherein the one or more mutation is a loss of function or partial loss of function mutation.

In a further aspect of the invention, there is a method for identifying and selecting a non-pea plant variety, seed or plant product that has improved digestion properties, the method comprising detecting in the plant genome or germplasm at least one polymorphism (i.e. an allele) in the SBE1 gene, wherein the polymorphism is a loss of function or a partial loss of function mutation.

In a further embodiment of any of the above-described methods, the method may further comprise producing a food composition or food product, vitamin or nutritional supplement from the selected plant or from the seed obtainable or obtained therefrom. In one embodiment, the food composition is the seed. In an alternative embodiment, the food composition may be a flour of any product derivable from a seed.

In each of these aspects and embodiments, the non-pea plant is preferably a plant which produces edible seeds, more preferably an edible seed which is suitable for processing into flour. The plant may be a legume, preferably a chickpea, alternatively a lentil, bean (optionally kidney, navy, pinto, black, cannellini), lupin bean, soy, or the like. Alternatively the plant may be a corn plant (Zea mays). In some embodiments the plant may be a grain plant; for example, wheat, barley, oat etc. In some embodiments the plant produces an edible root, for example parsnips; potatoes; sweet potatoes; yams; taro.

As shown herein, certain physico-chemical properties of foodstuffs can affect the amount of resistant starch available in a product. For example, the matrix structure of the foodstuff may influence how starch granule structure rearranges upon heating, and affects the resistant starch content available after cooking. Indeed, the present inventors unexpectedly found that the availability of resistant starch in some pea products increased after cooking. We hypothesise that certain preparation techniques and methods may contribute to an increase in resistant starch, in addition to the biological processes involved. Thus, in certain embodiments, the pea product (or non-pea plant seed product) may be processed with a method selected from cooking, hydration, milling, high pressure treatment, extrusion cooking or the like. In preferred embodiments, the processing method is selected to increase the resistant starch content in the product (potentially by decreasing the amount of non-resistant starch). For example, the processing method may comprise rough milling, to increase the size of resulting pieces and reduce cell wall breakage.

By “SBE1” is meant the starch-branching enzyme 1, which is an enzyme involved in starch synthesis. Specifically, the enzyme catalyzes the formation of the alpha-1,6-glucosidic linkages in starch by scission of a 1,4-alpha-linked oligosaccharide from growing alpha-1,4-glucan chains and the subsequent attachment of the oligosaccharide to the alpha-1,6 position. A partial or complete loss of function of this enzyme leads to a reduction in starch synthesis and an increase in the level of “resistant starch”. By “resistant starch” is meant starch that is largely unbranched amylose polymers that are resistant to digestion. In one embodiment, the SBE1 gene comprises or consists of a nucleic acid sequence that encodes the SBE1 amino acid sequence as defined in SEQ ID NO: 2 or a variant or homologue thereof. In a further embodiment, the SBE1 gene comprises or consists of a nucleotide sequence as defined in SEQ ID NO: 1 or a functional variant or homologue thereof. In another embodiment, the SBE1 gene is defined in Accession Number X80009, Version No. 1—i.e. GenBank X80009.1.

In a preferred embodiment, the loss of function mutation is introduced into the endogenous gene. In an alternative embodiment, a mutation is introduced into the SBE1 promoter to prevent expression of SBE1.

In one embodiment, the mutation is a partial or complete loss of function mutation. The loss of function mutation may be selected from one of the following mutation types:
1. a “missense mutation”, which is a change in the nucleic acid sequence that results in the substitution of an amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); plant genes contain the translation stop codons “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more amino acids, due to one or more codons having been added in the coding sequence of the nucleic acid;
4. a “deletion mutation” of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.
7. an “inversion” mutation, which is a one hundred and eighty rotation of a sequence of nucleic acid.

By “at least one mutation” is meant that where the SBE1 gene is present as more than one copy or homoeologue (with the same or slightly different sequence) there is at least one mutation in at least one gene. Preferably all genes are mutated.

In one embodiment, the loss of function mutation is a mutation in the C-terminus of SBE1. More preferably, the mutation is a 0.8 kb insertion that causes the loss of the last 61 amino acids of the SBE1 protein. This mutation is described in Bhattacharyya M K et al. 1990 et al, which is incorporated herein by reference.

The term “variant” as used herein with reference to any of the sequences defined herein refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates a SBE1 gene orthologue from other plant species. A homolog may have, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by SEQ ID NO: 2 or to the nucleic acid sequence shown in SEQ ID NO: 1. Suitable homologues can be identified by sequence comparisons and identifications of conserved domains. There are predictors in the art that can be used to identify such sequences. The function of the homologue can be identified as described herein and a skilled person would thus be able to confirm the function, for example when expression is knocked-out in a plant. Functional variants of SBE1 gene homologs as defined above are also within the scope of the invention.

In one embodiment, the mutation is introduced using targeted genome editing. That is, in one embodiment, the invention relates to a method and plant that has been generated by genetic engineering methods as described above, and does not encompass naturally occurring varieties or generating plants by traditional breeding methods.

Targeted genome modification or targeted genome editing is a genome engineering technique that uses targeted DNA double-strand breaks (DSBs) to stimulate genome editing through homologous recombination (HR)-mediated recombination events.

In a preferred embodiment, the genome editing method that is used according to the various aspects of the invention is CRISPR. Type II CRISPR is one of the most well characterized systems and carries out targeted DNA double-strand breaks in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art, such as http://chopchop.cou.uib.no/ it is possible to design sgRNA molecules that targets a SBE1 gene sequence as described herein.

In one example, sgRNA can be used with a modified Cas9 protein, such as nickase Cas9 or nCas9 or a “dead” Cas9 (dCas9) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017). Alternatively, the method may use sgRNA together with a template or donor DNA constructs, to introduce a targeted SNP or mutation, in particular one of the substitutions described herein, into a SBE1 gene. In this embodiment, the introduction of a template DNA strand, following a sgRNA-mediated snip in the double-stranded DNA, can be used to produce a specific targeted mutation (i.e. a SNP) in the gene using homology directed repair.

Once targeted genome editing has been performed, rapid high-throughput screening procedures can be used to analyse amplification products for the presence of a loss of function mutation in the SBE1 gene. Once a mutation is identified, the seeds of the M2 plant carrying that mutation are grown into adult M3 plants and screened for the phenotypic characteristics associated with the target gene SBE1. Loss of function mutants with at least one mutations in SBE1, and as a result, increased levels of resistant starch compared to a control can thus be identified.

In an alternative embodiment, the loss of function mutation is introduced using mutagenesis. These methods include both physical and chemical mutagenesis. In one embodiment, insertional mutagenesis is used, for example using T-DNA mutagenesis (which inserts pieces of the T-DNA from the Agrobacterium tumefaciens T-Plasmid into DNA causing either loss of gene function, site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999). In another embodiment, mutagenesis is physical mutagenesis, such as application of ultraviolet radiation, X-rays, gamma rays, fast or thermal neutrons or protons. The targeted population can then be screened to identify a loss of function mutant.

In another embodiment of the various aspects of the invention, the method comprises mutagenizing a target plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify a loss of function mutant.

In a further embodiment, the method used to create and analyse mutations is targeting induced local lesions in genomes (TILLING), reviewed in Henikoff et al, 2004.

In another aspect of the invention, there is provided a method of improving the digestion properties of a pea or of another edible seed or root, the method comprising reducing or abolishing the expression of a SBE1 gene, wherein expression of the SBE1 gene is reduced or abolished using gene silencing. “Gene silencing” is a term generally used to refer to suppression of expression of a gene via sequence-specific interactions that are mediated by RNA molecules—small interfering nucleic acids (siRNA) against SBE1. The degree of reduction may be so as to totally abolish production of the encoded gene product, but more usually the abolition of expression is partial, with some degree of expression remaining. The term should not therefore be taken to require complete “silencing” of expression.

In one embodiment, the siRNA may include, short interfering RNA (siRNA), double-stranded RNA (dsRNA), micro-RNA (miRNA), antagomirs and short hairpin RNA (shRNA) capable of mediating RNA interference.

Plants obtained or obtainable and seeds or edible roots obtained or obtainable from such plants by such method which carry a loss of function mutation in the endogenous SBE1 gene are also within the scope of the invention.

In one embodiment, the progeny plant is stably transformed with the CRISPR constructs, and comprises the exogenous polynucleotide, which is heritably maintained in the plant cell. The method may include steps to verify that the construct is stably integrated. The method may also comprise the additional step of collecting seeds from the selected progeny plant.

In a further embodiment, the method may further comprise at least one or more of the steps of assessing the phenotype of the genetically altered plant, specifically, measuring or assessing an increase in levels of resistant starch wherein preferably said increase is relative to a control or wild-type plant.

In another aspect of the invention, there is provided a genetically altered plant, part thereof or plant cell, wherein the plant comprises a complete loss of function mutation (also known as a null mutation) in the SBE1 gene. Preferably, the plant is characterised by an increased level of resistant starch. The level of resistant starch is increased compared to a plant lacking any mutation in the SBE1 gene or lacking a partial loss of function mutation. Preferably, the plant is a pea plant and the part is a pea or seed, as defined above. In other embodiments, the plant is a crop plant. More preferably, the plant is selected from beans (optionally kidney, navy, pinto, black, cannellini); squashes; chickpeas; corn; lentils; parsnips; potatoes; sweet potatoes; yams.

In a preferred embodiment, the mutation introduces a stop codon in SEQ ID NO: 1 such that a truncated protein is produced having no residual enzyme activity. In one embodiment, the mutation is a G to A substitution at position 680 from the start codon (ATG), which results in truncation of the protein at 226 amino acids (i.e. a W227* mutation). This mutation may be referred to herein as rl or r-l.

In another embodiment, there is provided a seed obtained from or obtainable from the genetically altered plant. In a further embodiment, there is provided progeny plant obtained or obtainable from the plant, as well as seed obtained or obtainable from the plant, and progeny obtained or obtainable from that plant. In a further embodiment, there is provided germplasm obtained or obtainable by the genetically altered plant of the invention.

In a preferred embodiment, the plant is a pea plant as defined above. The term “plant” as used herein encompasses whole plants, ancestors and progeny of the plants and plant parts, including seeds, fruit, shoots, stems, leaves, roots (including tubers), flowers, tissues and organs, wherein each of the aforementioned carry at least one of the herein described mutations. The term “plant” also encompasses plant cells, suspension cultures, callus tissue, embryos, meristematic regions, gametophytes, sporophytes, pollen and microspores, again wherein each of the aforementioned comprises the mutations as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant. In another aspect of the invention, there is provided a product derived from a plant as described herein or from a part thereof.

In a most preferred embodiment, the plant part or harvestable product is a seed or grain. Therefore, in a further aspect of the invention, there is provided a seed or grain produced from a genetically altered plant as described herein. Accordingly, in one aspect of the invention there is provided seed, wherein the seed contains one more of the genetic alterations described herein—specifically, the seed comprises one or more null mutations in SBE1. Also provided is progeny plant obtained from the seed as well as seed obtained from that progeny.

In an alternative embodiment, the plant part is pollen, a propagule or progeny of the genetically altered plant described herein. Accordingly, in a further aspect of the invention there is provided pollen, a propagule or progeny produced from a genetically altered plant as described herein.

A control plant as used herein according to all of the aspects of the invention is a plant which has not been modified according to the methods of the invention. Accordingly, in one embodiment, the control plant does not have one of the mutations in SBE1 described herein. In one embodiment, the control plant is a wild type plant. The control plant is typically of the same plant species, preferably having the same genetic background as the modified plant.

In a preferred embodiment, the mutation is introduced using targeted genome editing, such as CRISPR, as described above.

In another aspect of the invention, there is provided a method of increasing the level of resistant starch in a pea, the method comprising introducing a complete loss of function mutation in the SBE1 gene, wherein the mutation introduces a stop codon into the sequence of SEQ ID NO: 1 such that a truncated protein having no residual activity is produced. In a further embodiment, the mutation is introduced using targeted gene editing.

In one embodiment, the method comprises
a. selecting a part of the plant;
b. transfecting at least one cell of the part of the plant of paragraph (a) with at least one CRISPR construct or sgRNA molecule, wherein the CRISPR construct or sgRNA molecule targets the SBE1 gene and introduces the null mutation as described above;
c. regenerating at least one plant derived from the transfected cell or cells;
d. selecting one or more plants obtained according to paragraph (c) that show at least one null mutation in the SBE1 gene.

In one embodiment, the method may comprises obtaining a DNA sample from a transformed plant and carrying out DNA amplification to detect the at least one null mutation in the SBE1 gene. In a further embodiment of any of the methods described herein, the method may further comprise at least one or more of the steps of assessing the phenotype of the genetically altered plant, measuring at least one of increased levels of resistant starch. In other words, the method may involve the step of screening the plants for the desired phenotype.

Transformation methods for generating a genetically altered plant of the invention are known in the art. Thus, according to the various aspects of the invention, a CRISPR construct as defined herein is introduced into a plant and expressed as a transgene. The construct is introduced into said plant through a process called transformation. The terms “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The CRISPR construct may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

Transformation of plants is now a routine technique in many species. Advantageously, any of several transformation methods may be used to introduce a CRISPR construct into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microinjection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The method may further comprise regenerating a genetically altered plant from the plant or plant cell wherein the genetically altered plant comprises in its genome at least one null mutation, particularly the null mutation defined above in a SBE1 gene, and obtaining a progeny plant derived from the transgenic plant, wherein said progeny exhibits at least one the mutation in the SBE1 gene and shows an increase in levels of resistant starch compared to a wild-type or control plant.

In a further embodiment, the methods comprise generating stable T2 plants preferably homozygous for the mutation (that is a mutation in at least one SBE1 gene sequence).

In another aspect of the invention, there is provided a food or feed composition derived from the genetically altered plant or seed, as described above. The food composition may be flour prepared from the seeds of the invention. The flour may be further combined with other flours or ingredients. The term “flour” or “pea flour” is meant the product obtained by dried pea seeds. In another aspect of the invention, the plants, peas or seeds of the invention may be used to prepare a vitamin or nutritional supplement.

Accordingly, in a further aspect of the invention, there is provided a method for producing a food composition, vitamin or nutritional supplement, the method comprising producing a genetically altered plant, as described above and producing a food composition, vitamin or nutritional supplement from the plant, peas or seeds.

In a further aspect of the invention, there is provided a method of providing glycaemic control, the method comprising administering a diet of the food composition, vitamin or nutritional supplement to an individual in need thereof.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appIn cited documents”) and all documents cited or referenced in the appIn cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example I: rr Starch Genotype and Food-Structure Reduce Postprandial Plasma Glucose and Serum Insulin

Here, we investigate the impact of food structure, carbohydrate type, and the bioaccesibility of starch in the gastrointestinal environment (gastric and duodenal digestion and colonic fermentation) on PPG and gut bacteria related to glucose metabolism. We used mature seeds of pea (Pisum sativum L.), as a model food in a series of experiments spanning in vitro laboratory to metabolic studies in humans.

There is systematic evidence that non-oil-seed pulses, of which pea is a member, have a significant impact on long term glycaemic control [8]. Additionally, the genetic variation available in pea, provides an opportunity to investigate the impact of starch assembly on digestive processes. As the physical state of food can be a significant determinant of postprandial glycaemia, using pea flour enables the study of this phenomenon in some depth.

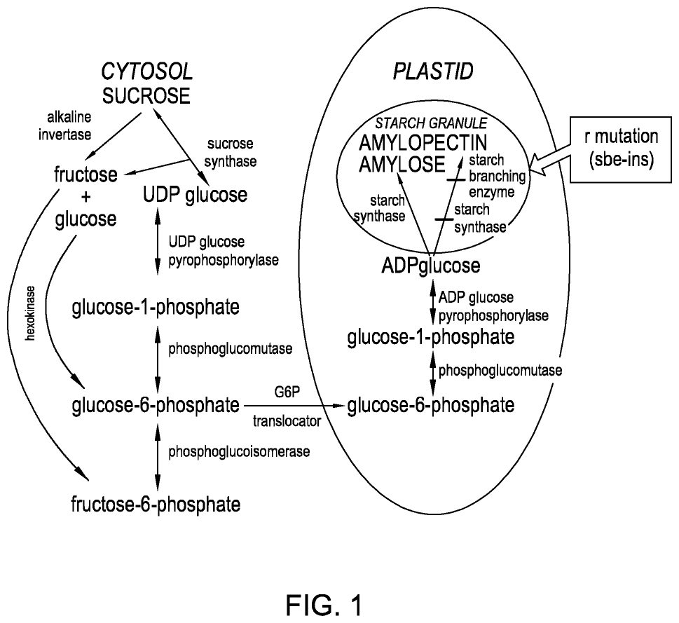

We used two near-isogenic pea lines, which are genetically identical except that one (BC1/19rr) carries a natural mutation in the starch branching enzyme I gene (SBEI) [9]. In BC1/19RR, the wild-type or control line, SBEI makes a major contribution to the amylopectin fraction present in seeds, where the enzyme is active within the plastids of the cotyledonary cells (FIG. 1). The naturally occurring mutation in the sbel gene (BC1/19rr) disrupts the carboxy-terminal region of the protein, affecting the structure of the starch and other seed phenotypic traits [9, 10]. In the mutant line, the majority of the starch which is synthesised has been dubbed ‘resistant starch’, reflecting its largely unbranched amylose polymers and resistance to digestion. This naturally occurring mutation is unique in rr peas. However, mutations in SBE exist in other species but these are mainly induced mutations, such as those in rice [11], durum wheat [12], bread wheat [13], rice and potato [15].

In this work, we compared BC1/19RR wild-type and mutant BC1/19rr peas to examine the effects of genetic alterations to starch structure on digestion parameters (using in vitro oral/gastric and duodenal simulated digestion models) and associated health outcomes (by performing experiments in vivo, in human volunteers). We investigated the effects of processing and food structure by generating pea flour and producing pea-derived food products where processing had disrupted of the cell wall. The acceptability of these products and the relative effects on glucose metabolism were tested in a sample of healthy volunteers with no compromised glycaemic control.

### Results

### Food-Structure and rr Starch Genotype Attenuates Postprandial Glycaemia

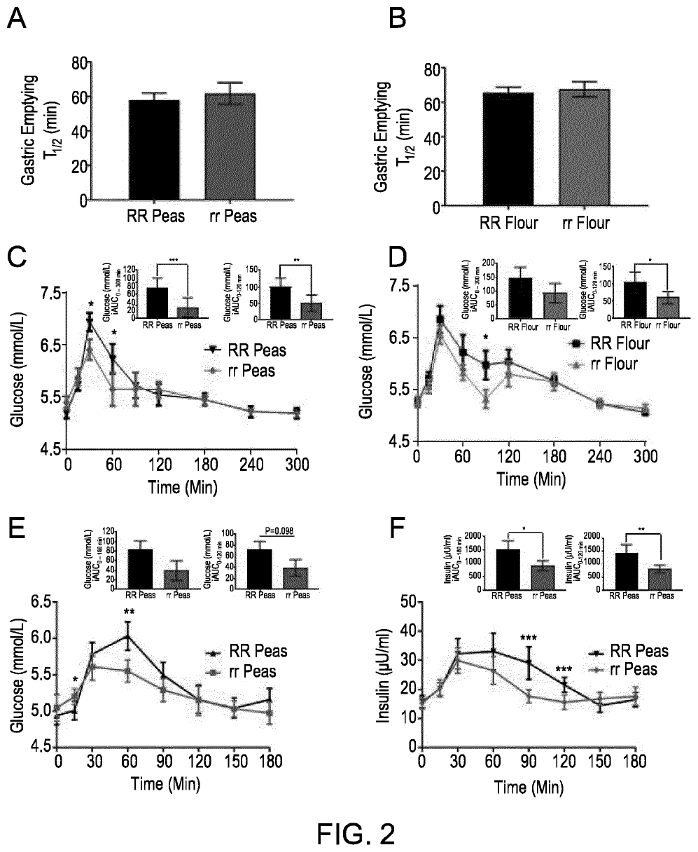

In a randomized, controlled, double blind, cross-over trial (trial 1), 10 healthy volunteers were fed a cooked, mixed meal containing 50 g dry weight of pea seeds or flour from two pea genotypes (RR and rr, providing 31% and 26%, of the total carbohydrate content of the meal, respectively; see Methods and Supplementary Table 1). There was no significant differences in solid phase gastric emptying, assessed by the [¹³C] octanoic acid breath test [17], between the rr vs RR pea seeds or flour groups (p=0.49 and p=0.59, respectively; FIG. 2A, B), as determined by Ty, (half-emptying time), indicating that volunteers began digesting the different test meals in a similar time course. There was a significantly lower PPG between the rr and RR pea seeds and between the rr and RR flour groups (effect over time, p=0.02 and p=0.04, respectively; FIG. 2C, D).

In a second randomized, controlled, double blind, cross-over trial (trial 2) 12 healthy volunteers were fed 50 g dry weight of pea seeds or flour from two pea genotypes (RR and rr,) as a cooked product (rather than a mixed meal; Methods and Supplementary Table 2). For pea seeds group, plasma glucose and serum insulin concentrations were significantly lower after consumption of rr compared to RR (effect over time, p=0.02 and 0.001 respectively) (FIG. 2E, F). There was no effect on glucagon-like peptide 1 (GLP-1) (FIG. 2G). The RR pea seeds consumption led to a higher release of gastric inhibitory polypeptide (GIP) compared to rr (effect over time, p=0.01) (FIG. 2H). For flour, a lower PPG response occurred after consumption of rr compared to RR (effect over time, p=0.06) (FIG. 2I). Serum insulin incremental area under the curve 120 min (iAUC_{0-120 min}) was significantly reduced by 37% (p=0.04) for rr compared to RR flour (FIG. 2J). During the first 15 min post ingestion, there was a significantly higher peak in GLP-1 concentrations observed for RR compared to the rr group (p=0.001) (FIG. 2K). There was also a significantly higher iAUC_{0-120 min} GIP for RR compared to rr (p=0.02) (FIG. 2L).

Changes in food structure, induced through processing whole seeds to flour within genotype, showed profound effects on PPG and serum insulin. In both RR and rr, processing to flour produced a significantly larger glucose and insulin response over 180 min (Supplementary FIG. 1A-D).

Together, these data demonstrate that both starch genotype and food structure have an impact on postprandial glycaemia.

### Example II: Impact of Pea Structure and Starch Assembly on Starch Digestion

A series of in vitro studies elaborated physico-chemical mechanisms of starch digestion and nutrient bio-accessibility in RR and rr pea seeds and flour.

### Starch Digestibility

The total starch contents of pea seeds and flour were determined at raw, post-cooking and post-simulated digestion (oral, gastric/small intestinal conditions) stages (FIG. 3A). Starch digestion in cooked pea seeds was 60% for RR and 24% in rr (FIG. 3A) indicating that the starch in rr was less digestible by the upper gastrointestinal enzymes versus RR pea seeds (p<0.0001), and corroborating FIG. 2 findings.

After cooking, the portion of analytically resistant starch (ARS) content (that is, the estimated proportion of resistant starch using standard laboratory methods), based on the AOAC 2002.02 method [18], decreased in RR pea seeds (p<0.05) but increased in the rr after cooking and digestion (p<0.01). We observed no statistical significant difference between the RR and rr flour (FIG. 3B).

¹³C cross polarized magic angle spinning (CP-MAS) NMR was used to establish the helical structure of the starch in uncooked and cooked pea seeds and flour, a key determinant of its resistance to digestion (FIG. 3C & Supplementary FIG. 2) [19, 20]. The starch in the uncooked RR line presented a 35% double helical structure, in both pea seeds and flour (indicating that the starch was not significantly altered during milling), whereas the rr line had a lower proportion of double helices (19%) (FIG. 3C). Following cooking of the flours the starches from both genotypes (RR and rr) fully gelatinized, having less than 10% double helical order. Starch from pea seeds of the RR genotype only partially gelatinised, with a small decrease in double helical order (from 34% to 27%) (p<0.05), whereas in the rr genotype there was an increase in double helical order observed following cooking (from 20% to 31%) (p<0.01). This difference in gelation behaviour suggests that the matrix structure (flour vs peas) influences how the starch granule structure rearranges upon heating, and controls the ARS. This concurs with the observed PPG and serum insulin concentrations between the RR and rr flour compared to RR and rr pea seeds (FIG. 2).

We hypothesized that in the spatially and water limited environment of the plant cell, the starch undergoes structural rearrangements that are different to those in the flour, leading to higher levels of ordered structures in the cooked pea seeds relative to cooked flours. These observations may be attributed to differences in the chain length distribution of the starches (FIG. 3D), where the rr starch has far fewer short (Rₕ<4 nm) amylopectin chains and a greater proportion of longer amylose chains. A higher proportion of longer amylose chains which limits swelling of the starch (FIG. 3F) alters recrystallisation following cooling (FIG. 3C) and leads to a greater proportion of starch escaping small intestinal digestion and reaching the colon to be ferment by resident gut bacteria.

### Food-Matrix and Processing and Digestion

Pea seed fragments (>2 mm) formed following simulated oral phase ‘chewing’ survived in vitro digestion intact. Therefore, the particle size was measured post-gastric and intestinal phases. rr digesta contained more larger particles (>700 μm) compared to RR (FIG. 3E) The digesta from RR contained a higher proportion of smaller particle (<250 μm) than rr (such as individual cells and free starch), suggesting that the RR tissue was more friable than for rr. The particle size did not change significantly during simulated digestion, so the major impact on the structure was from cooking and simulated chewing.

Compression experiments to understand fracture profiles of cooked pea seeds demonstrated that force-deformation curves were higher in rr compared to RR at 1 mm/s (p<0.0001) but were similar in both genotypes when compression rate was increased to 15 mm/s (FIG. 3H). The rr pea seeds fractured into larger particles of tissue in contrast to RR, where they appeared to breakdown completely (FIG. 3H). Although the moisture content for rr was higher than for RR cooked seeds (60% vs 45%), it was noted that the physical appearance of compressed seeds was different; rr seeds tended to split while RR seeds were crushed (FIG. 3H). The greater resistance of the rr seeds to deformation could be attributed to known differences in cell wall properties, structural differences in the intracellular starch and/or protein and/or degree of hydration of plant tissue components [22].

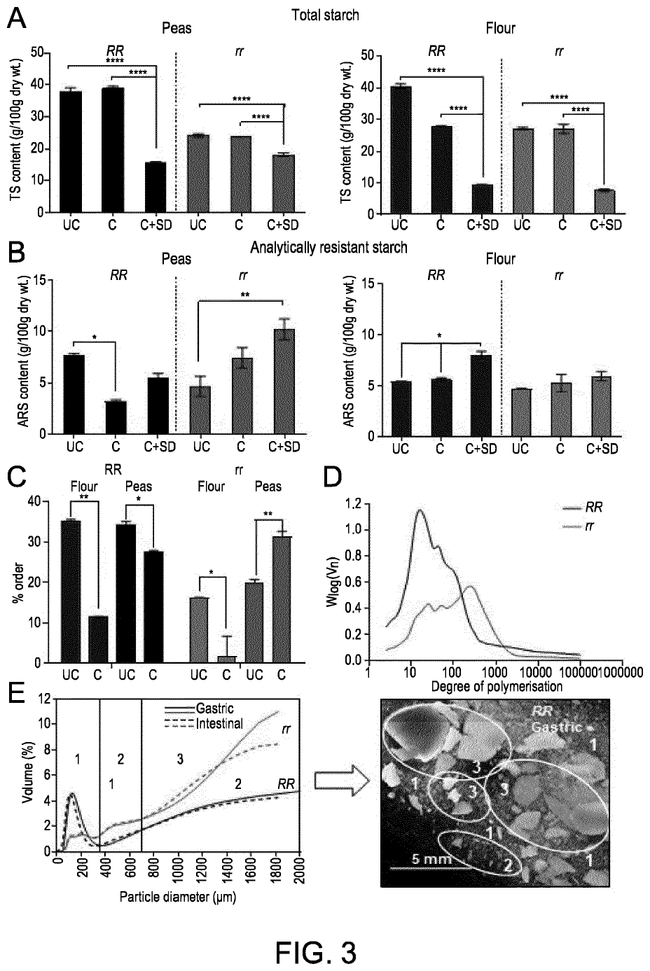

Micrographs of flour and pea seed sections demonstrated the impact of cooking (FIG. 3F, G) and simulated digestion (Supplementary FIG. 3) on the cellular structure and starch morphology. Micrographs of flour demonstrated the influence of cooking on pea starch following the loss of the pea matrix by milling into flour. The raw starch granules of rr were very different in morphology to those of the RR genotype (FIG. 3F), composed of a mixture of simple and compound granules due to high amylose content [23]. Starch from RR flour appeared to be almost fully gelatinized after cooking (FIG. 3F) and was no longer visible after digestion in vitro (Supplementary FIG. 3). Together with the shorter chain lengths in RR (FIG. 3D), this may explain the loss of total starch in RR flour following cooking, as the shorter chains are likely to have leached out of the granules, avoiding detection by the starch assay. However, intact starch granules from rr flour persisted throughout cooking, showing many intact, non-gelatinised granules (FIG. 3F) and following digestion in vitro (Supplementary FIG. 3). After simulated duodenal digestion, however, the remaining rr flour granules were stained pink rather than blue. After cooking, RR starch granules gelatinized to a greater extent than those of rr (FIG. 3G), where the starch granules appeared less swollen. In the RR peas, starch appeared to be hydrolysed in some cells and not in others, after digestion in vitro (Supplementary FIG. 3). On the other hand, starch granules in rr pea cells, that were less swollen after cooking, appeared undigested within the cells (Supplementary FIG. 3). Scanning electron micrographs (Supplementary FIG. 4) of uncooked and cooked pea seeds confirmed the extent of starch gelatinization within cotyledon cells seen with light microscopy and also suggested some morphological differences between RR and rr.

The results show that there are at least two main factors which influence starch digestibility in the pea samples studied. Firstly, the matrix structure, where intact plant cell walls encapsulate the starch, act as enzyme barriers and also hindering gelatinization of intracellular starch by reducing access to water; and secondly the intrinsic resistance of the starch granule, with the higher ARS content of the rr genotype making it more resistant to digestion. Even though the rr starch in the flour lost much of its order, the morphology of the rr starch granules was affected less than RR starch following cooking. Furthermore, the tissue matrix affected fracture properties such that chewing produced larger particles for rr pea seeds and thus more intact cells acting as a barrier to digestion.

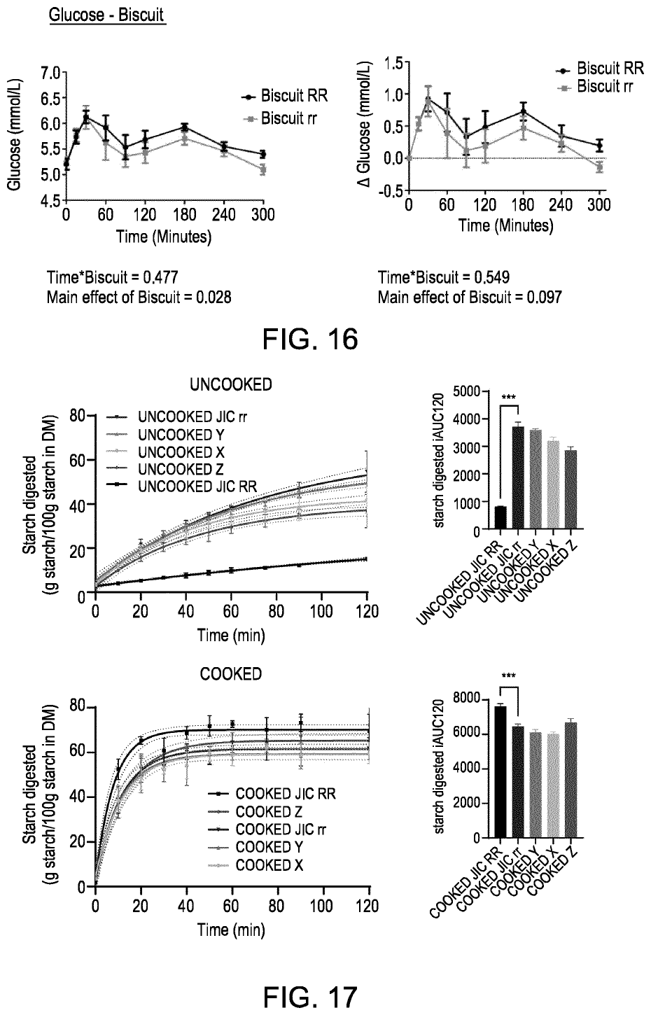

Reference is also made to FIG. 17, which compares starch digestion rates of cooked and uncooked milled pea cotyledon flours from rr and RR peas, as well as control commercial varieties. The data demonstrates that in the uncooked state, rr pea starch is significantly more digestible than RR pea starch, and that once cooked, the RR pea starch is significantly more digestible than the rr pea starch. Differences in starch amylolysis profiles from rr vs RR pea starch provide a reasonable predictive indication of relative differences in postprandial glycaemic responses. Hence, the combination of rr genotype and processing (in this case, cooking) can serve to achieve a desired postprandial glycaemic response from a foodstuff.

Without wishing to be bound by theory, we surmise that the difference between rr and RR starch granules is that the starch granules in rr peas are smaller and appear cracked, so the amylopectin fraction is more accessible in the rr, leading to more rapid digestion. When cooked, the wild type is digested more quickly than the rr. Microscopy evidence suggests that during cooking, in the rr pea some amylose leaches out of the granules and forms a protective layer around them and slows digestion, whereas in RR peas, the starch granules are fully gelatinised and are more rapidly digested.

### Detailed Experimental Method for FIG. 17

Step 1: Whole peas were used; prior to commencing experimental work, the pea varieties; ‘Commercial variety X’, ‘JIC RR’, ‘Commercial variety Y’, ‘JIC rr’, and ‘Commercial variety Z’, were coded A, B, C, D, E, respectively, by an independent researcher so that the researcher undertaking the analysis was ‘blinded’. Cotyledons were obtained from whole peas by removing the testa and embryo: Whole peas (40 g) were soaked in water overnight. The testa and embryo were then manually removed from the peas to obtain pure cotyledon (starch-containing) tissue.

Step 2: Pea cotyledons were then dry-milled to obtain a sub-cellular flour for subsequent analysis: Pea cotyledons were then left to dry at 35° C. overnight. The dehulled pea cotyledons were blended in a KRUPS F20342 Coffee Grinder for 1 min in six periods of 10 s with a pause of 5 s in between each period. The milled material was sieved on an Endecott analytical sieve with 150 μm aperture to obtain milled cotyledon flour sub-cellular particles <150 μm. Milled particles retained on the 150 μm were re-blended for up to 3 min then re-sieved. This dry-milling process breaks open the majority of pea cells in the cotyledons.

Step 3: Starch amylolysis assays were performed on uncooked and cooked pea cotyledons under a fixed enzyme-starch ratio, as per (Edwards et al., 2020, Edwards et al., 2019, Edwards et al., 2018; references [41], [42], [27]): A precise mass of uncooked pea cotyledon flour was added to 15 mL Falcon tubes, in which the mass of flour required per tube ranged from 100 to 150 mg fresh weight, and was the precise amount of flour that contained 50 mg starch. The mass of sample needed was calculated from the total starch (measured by K-TSTA-100A Megazyme Total Starch alkali method for starch containing resistant starch) and moisture content (change in weight following oven-drying at 103° C.) of each sample—This approach accounts for differences in total starch content between pea varieties and thereby enables the intrinsic starch digestibility to be studied.

Flour samples were then suspended in 10 mL of phosphate buffered saline (PBS, Oxoid, pH 7.4 at 37° C.) by vortex mixing. To prepare cooked flour samples, the samples were hydrothermally processed in a water bath at 90° C. for 10 min with vortex mixing every minute, at this stage. For uncooked samples, the suspensions were put in the platform (prs 26) of a PTR-35 Grant-bio rotator at the appropriate spacing for simultaneous sampling of 3 tubes with a multi-pipette. For equilibration, the cooked or uncooked suspensions were mixed end-over-end at 60 rpm inside a 37° C. incubator for 15 min (E24 Excella, New Brunswick Scientific).

A dilution of porcine pancreatic α-amylase (EC 3.2.1.1 A6255, Sigma-Aldrich) was prepared in phosphate buffered saline (PBS) to obtain a working solution with amylase activity of 100 U/mL. The rotation was halted to allow the samples to settle for 15 s, then aliquots of 100 μL were withdrawn and stopped in an equal volume of 0.3 M Na2CO3. Triplicate additions of amylase working solution (100 U/mL) were made at 15 s intervals to start the digestion. The digestions were sampled at 10, 20, 30, 40, 50, 60, 75, 90 and 120 min. The stopped aliquots were centrifuged at 15,000 g for 5 min at 20° C. Heraeus Pico, Thermo Scientific) and 130 μL of the supernatants were retained.

Step 4: Starch amylolysis products were measured by PAHBAH assay: Samples collected during starch amylolysis were appropriately diluted with deionised water and 100 μL of the diluted sample transferred to a 1.5 mL Eppendorf® Safe-lock™ tube, to which was added 1000 μL freshly prepared ‘PAHBAH working reagent’ (250 mg p-hydroxybenzoic acid hydrazide dissolved in 4.75 mL of 0.5 M HCL, and made up to 50 mL with 0.5 M NaOH). These tubes were vortex mixed and secured in a Nalgene® floating rack and incubated at 100° C. for 5 min, then equilibrated for 10 min at room temperature before transfer to cuvettes and absorbance measurement (λ=405 nm) in a spectrophotometer (Biochrom Libra). Standards containing known concentrations of maltose (0-900 μM) were prepared in 1.5 mL Eppendorf® Safe-lock™ and reacted with PAHBAH reagent as described above, and these were included with every analysis. This enabled calculation of the concentration of reducing sugars (as maltose equivalents) at each time point. The maltose product concentrations were divided by the initial starch mass to express the percentage (%) of total starch digested at each time point.

### Results & Discussion

Data from the starch analysis of cotyledons in the uncooked state (see Supplementary Table 16) shows that JIC RR cotyledons contained significantly higher amounts of total starch (52.9 g starch/100 g DM) and significantly higher amounts of resistant starch (13.8 g resistant starch/100 g DM) than any of the other pea varieties studied (group mean for pea varieties except RR was ˜41 g TS/100 g and 6.75 g RS/100 g DM). This means that in the uncooked RR pea cotyledon, approximately 26% of the starch is analytically resistant starch, while in the other pea varieties between 15.1 and 17.6% of the starch was resistant.

The same pattern is evident in the starch amylolysis assays (FIG. 17, top row)—uncooked JIC RR pea is most resistant to starch amylolysis, and shows less starch digestion than the other pea varieties. With this method, it is possible to resolve significant differences between pea varieties from the time-course data. Notably, in the uncooked state, starch digestibility in cotyledons from the Commercial variety Y was not significantly different from the JIC rr sample.

The observations made on uncooked samples are of little relevance to human nutrition considering that peas require some form of processing prior to consumption. The starch amylolysis curves obtained for cooked samples (FIG. 17, bottom row) provide a more useful basis for selecting varieties that are likely to give a lower glycaemic response. From starch amylolysis assays of cooked pea cotyledons, it was evident that once cooked, the rr pea cotyledon showed significantly lower starch digestibility compared with the RR pea (p<0.001), which is consistent with previous work, including the previous publication Edwards et al 2018, [27]. Thus, a combination of genotype and processing can be used to determine expected post prandial glycemic response.

### Example III: rr Genotype Results in Reduced Small Intestinal Glucose Release in Humans

We investigated the impact of the rr mutation on duodenal glucose release in humans by intubating the small intestine and stomach of 12 healthy volunteers via the nasal route (trial 2). After consumption of pea seeds small intestinal glucose concentration for the RR group, at 30 min, was 3.77±2.28 mmol/L which was nearly two-fold higher than for the rr group at the same time point (1.92±2.21 mmol/L) (FIG. 4A). We found that in the rr group, the area under the curve 120 min (AUC0-120 min) for small intestinal glucose concentrations was significantly lower compared to the RR group (p=0.02; FIG. 4B). We found no statistically significant differences in the small intestinal glucose responses between flour groups from the two pea genotypes (FIG. 4C, D).

### Gastric and Small Intestinal Metabolic Profiles for Whole Pea Seeds

The gastric and small intestinal metabolic profiles of the aspirated samples were assessed using an untargeted metabolic profiling approach by Proton Nuclear Magnetic Resonance (¹H-NMR) spectroscopy. Significant differences were found in the metabolic profiles of gastric samples of the RR and rr pea seed groups (FIG. 4E). Signals corresponding to the group of metabolites (full list Supplementary Tables 3, 4), including amylopectin/maltotriose/maltose, were significantly higher for the RR samples at 30 min post ingestion compared with those of rr (p=0.0004, Q=0.002).

We found statistically significant differences in glucose release rates, at 60 min post ingestion between the two pea groups (FIG. 4F). RR pea seeds group resulted in higher glucose release compared with rr group (p=0.001, Q=0.01) (Supplementary Table 5). The data suggests that leaching of amylopectin/maltotriose/maltose from the RR seeds makes them more susceptible to early digestion and release of glucose.

### Gastric and Small Intestinal Metabolic Profiles for Pea Flour

1H-NMR metabolomic analysis of gastric flour samples indicated differences between the two pea genotypes (FIG. 4G). Similar, to pea seeds, we found higher amylopectin/maltotriose/maltose in the gastric content of the RR flour group at 15- and 30-min post ingestion (p=0.00004, Q=0.0019/p=0.0001, Q=0.0006 respectively; Supplementary Table 6).

In the small intestinal samples, the RR flour group showed higher glucose concentrations at 30 min compared to rr (p<0.001, q<0.001) (FIG. 4H). We also observed higher release of sucrose and alanine for rr flour compared to RR (Supplementary Table 7).

α-amylase Permeability In-Vitro and Ex Vivo

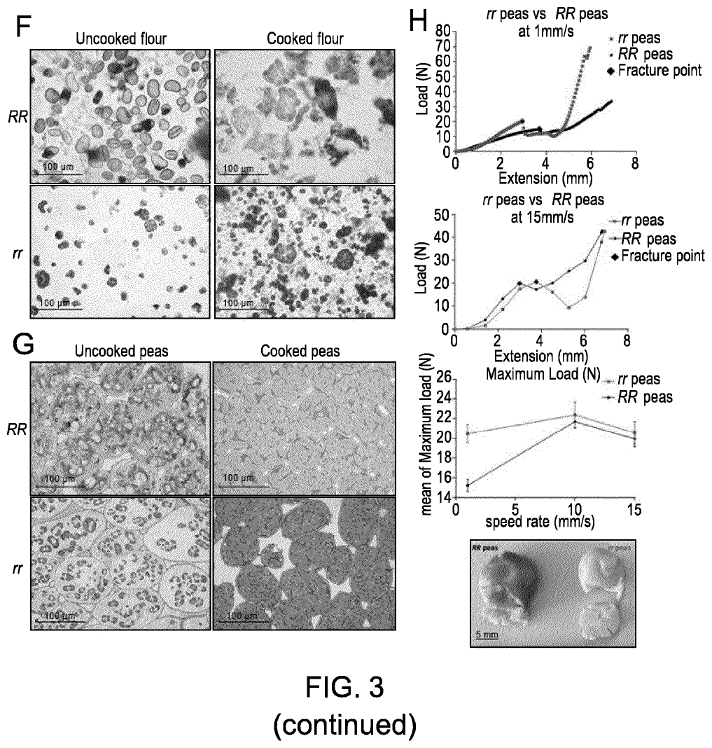

Time course data from confocal microscopy showed that, within 10 min, FITC-amylase had diffused into the cell walls of both rr and RR pea seeds (FIG. 4I) but not yet passed into the intracellular space. Further ingress of the enzymes into the intracellular space was slow, as captured by the diffusion constant (given by fluorescence intensity·nm²·min⁻¹; 6.19×10⁻¹⁰ for rr and 1.23×10⁻⁹ RR (summarised in histogram, Supplementary FIG. 5), and there was heterogeneity in plant cells obtained from RR pea seeds, as seen in the time course data (FIG. 4I). For flour group, the amylase had bound to the surface of starch granules within 5 min and seemed to progressively erode the starch granules over time (FIG. 4J). These experiments indicate that the encapsulation of starch by the cell wall obstructs interaction with amylase enzyme. The diffusion of amylase across the intracellular space appeared to be slower for rr flour than RR overall.

Using the small intestinal digesta from the study in vivo we performed experiments ex vivo aiming to understand the cell wall permeability to α-amylase (AA). In both RR and rr peas the diffusion of AA-FITC into cells was progressive with time and the diffusion of AA in rr pea samples was slower than in RR (FIG. 4K). Uptake of AA-FITC into RR and rr pea flour was almost immediate (FIG. 4K).

### Example IV: rr Genotype Increases SCFA Production

We grew both RR and rr pea seeds in a ¹³CO₂ enriched environment in a hermetically sealed greenhouse, producing pea starch with an enrichment of ˜0.2 atom percent ¹³C above natural abundance and assessed labelled metabolites in plasma and urine.

Volunteers (n=10) took part in a third randomized, controlled, double blind, cross over trial (trial 3) to investigate ¹³C glucose and ¹³C SCFA appearance after 50 g dry weight RR and rr pea seeds or flour consumption included in a mixed meal (Supplementary Table 8). The time course and AUC for exogenous ¹³C postprandial plasma glucose indicated significantly higher concentrations after consumption of the RR as opposed to the rr pea seeds test meal (FIG. 5A, B). There was no significant effect observed when comparing RR and rr flour for exogenous postprandial ¹³C plasma glucose and total AUC_{0-480 min} (FIG. 5C, D). These observations support the PPG results observed in the FIG. 2 (trial 2).

We measured fractional recovery of ¹³C SCFA (acetate, propionate and butyrate) in 24 h urinary collections. ¹³C acetate excretion did not result in significant differences between RR and rr in either the seed or flour groups (p=0.65). ¹³C propionate and ¹³C butyrate output was significantly higher after consumption of rr, either seeds or flour (p=0.01, p=0.03, respectively) (FIG. 5E). This suggests that, with rr test meals, carbohydrate was not fully digested in the small intestine and more was delivered to the colon where it was fermented by the gut microbiota. We investigated whether or not changes in the stool gut microbiome might occur over 24 h but there was no difference in gut microbiome diversity between the pea genotypes for either test meal (FIG. 5F).

These data suggest that the main effect on glucose absorption is the structural barrier of the pea seeds which is enhanced by the rr genotype. However, the SCFA production highlights that the starch from the rr flour was not full digested in the small intestine. There was no evidence of an acute effect on stool microbiota diversity although we observed an increase in SCFA production with the rr genotype in both pea seeds and flour.

### Example V: Effect of RR Versus Rr Pea Seed Products Consumption on Glycaemic Control Independently of the Food Matrix

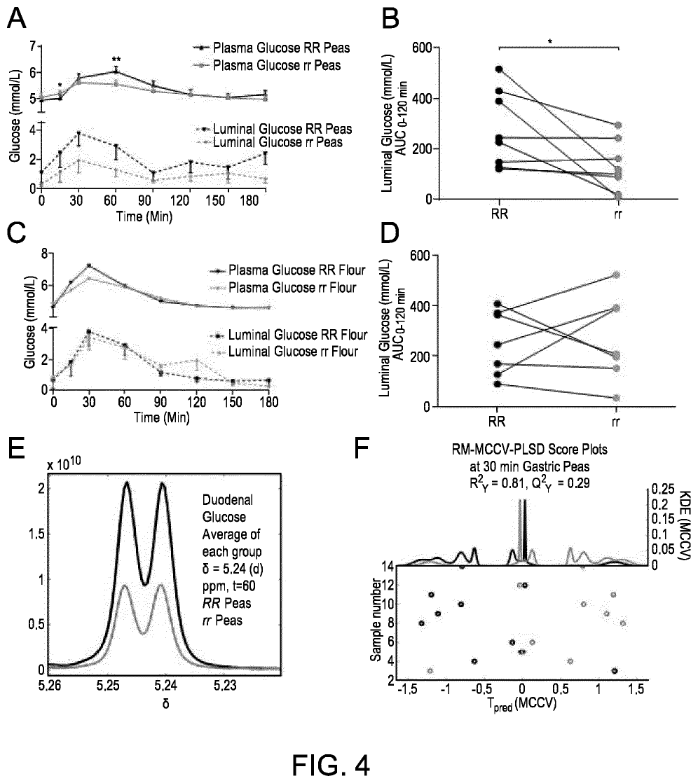

To understand the effects of the pea genotype independently of the food matrix on the PPG and gut bacteria we used a randomized, double-blind, 4 phase crossover control trial (trial 4) in 25 healthy volunteers aged 40-70 years (Supplementary Table 9, Supplementary FIG. 6 for volunteer characteristics & consort diagram). Volunteers were provided with mushy-peas and pea-hummus from both the RR and rr lines to consume for 28 days in random order. The nutritional content of the products can be found in Supplementary Table 10. All measurements were performed at baseline (0 days) and follow up (28 days) after a 12 h overnight fast. We assessed the effects of repeated pea consumption exposure on stool gut bacteria, glucose metabolism, as well as GLP-1 and lipids. A full summary of all outcome variables can be found in Supplementary Table 11.

### Glucose Metabolism

During the experimental visits, at baseline and follow up, volunteers did not receive the interventional pea-derived products but a mixed meal tolerance test as the aim of the study was to examine the chronic effect of products consumption. No statistically significant differences in markers of plasma glucose and serum insulin within or between groups were observed (FIG. 6A-D). We observed no differences in B-cell function and insulin sensitivity, using the Homeostatic Model Assessment 2 (HOMA 2), within or between groups. A possible explanation for this outcome might be the differences in the study design from that of the acute studies where volunteers consumed the pea seeds or flour. The rationale behind the study design here was that the chronic exposure to the intake of rr pea-derived food products would change the microbiota and lead to metabolic changes that would affect PPG and insulin responses, what has been termed as a “second meal effect”.

Gut Microbiome 16S rRNA Gene Sequencing

Nonmetric multidimensional scaling plot (NMDS) indicated no statistically significant effect in the clustering within or between RR or rr interventions (within RR: p=0.83, within rr. p=0.92; between interventions: p=0.92) (FIG. 6E). Due to high inter-individual variability posed by changes in baseline gut microbiota and the habitual diets between volunteers, we decided to examine the data as paired samples per volunteer and looked specifically for gut bacteria related to insulin resistance. Within the rr intervention group, at genus level, there was a decrease in the relative proportion of Bacteroides (p=0.04) and an increase in the relative proportion of Bifidobacterium (p=0.007) (FIG. 6F). Within the RR intervention group, results indicated a statistically significant decrease in Lachnospiraceae and Ruminococcaceae (p=0.01, p=0.004 respectively), which are known as starch degraders. Between groups (RR and rr interventions), a statistically significant decrease in the relative abundance of Collinsella was observed after 28 days of rr supplementation (p=0.03). Previous studies have reported increased levels of Collinsella in individuals suffering from T2D [24].

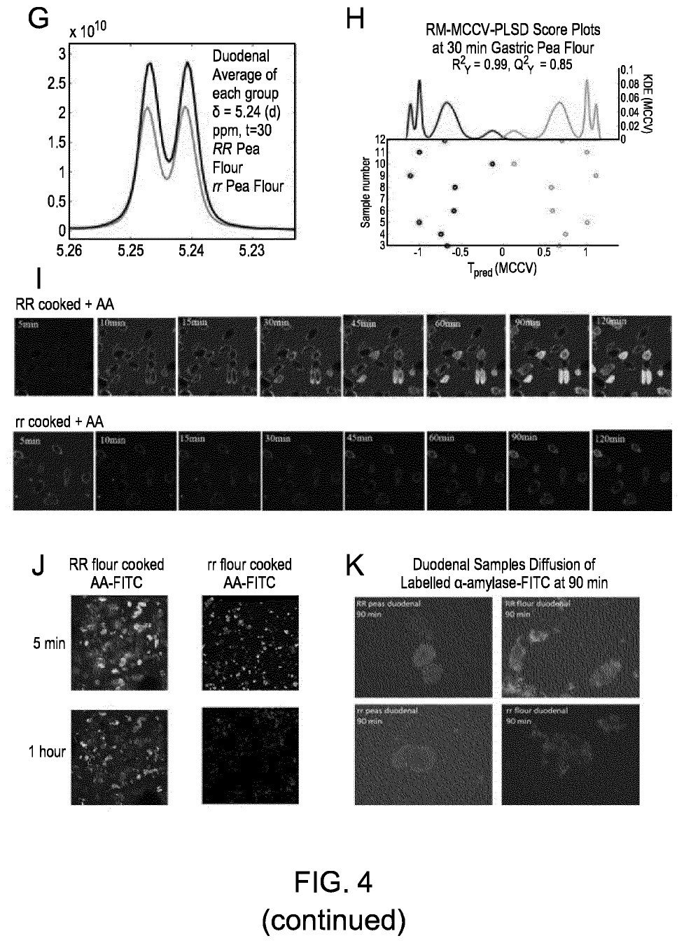

Despite observing differences in gut bacteria, there was no effect on glucose and insulin responses even though controlling for adherence to the interventions by measuring trigonelline, a validated pea biomarker (Supplementary Notes and Supplementary FIG. 7). We suggest that the food matrices of pea seeds have an acute impact on PPG, as seen in acute studies. The findings support the concept, that whilst dietary interventions may promote changes to gut bacterial populations, this may not automatically translate into improvements to glucose metabolism in “free-living” individuals.” In an ideal situation we would have run two experiments by including the pea seeds/products and by just performing a mixed meal tolerance test (as the one we did). However, this is difficult for many possible reasons such as blood volume loss.

### Discussion

Utilising the mutation which results in a defective starch branching enzyme and an increase in resistant starch in rr compared with RR wild-type pea genotypes, we demonstrate that pea seeds and flour from the rr genotype, significantly impact glucose and insulin homeostasis compared to the RR wild type. This is most marked when the cellular structures remained intact during digestion suggesting that bio-accessibility of amylase to the starch structures in the cell play a major role, an effect observed previously in intact wheat endosperm with a concomitant decrease in PPG [25]. The impact of rr flour on post prandial blood glucose was less marked than the pea seeds. This may be due to two factors; firstly, the initial rate of amylase digestion is the same between the genotypes but decreases in the rr genotype over time [26]. This is due to the starch granule of the rr seeds having an outer layer of amorphous starch and is rapidly digested, as in the RR genotype, but also has an inner core of crystalline starch [27]. Secondly the transit time in the duodenal space in humans is less than two hours, which is possible not long enough of the crystalline resistant starch inner core of the rr genotype to make a difference to the glucose availability in the duodenal space between the two genotypes. This observation is supported by one of the unique experiments reported in this manuscript: the direct measurement of glucose in the duodenum of humans. Here we show that the availability of carbohydrate in the small intestine directly relates to plasma glucose concentrations. The more resistant starch structures in the rr pea seeds led to lower duodenal and lower PPG with a greater transfer of carbohydrate to the large bowel.

The higher availability of glucose in the duodenum from RR pea seeds is associated with an increased release of GIP which may explain the higher insulin levels. Duodenal glucose infusions in humans have shown similar findings with high concentrations and flow rates of

glucose in the duodenum increasing both GLP-1 and GIP concentrations in the plasma [28]. Although we did not observe a significant difference in direct measures of duodenal postprandial glucose in the flour group, the NMR analysis suggests a higher duodenal glucose at 30 min which does coincide with the higher GIP and GLP-1 concentrations in the RR flour. We conclude that postprandial insulin concentrations are higher after the consumption of RR pea seeds and flour and this is driven through a higher availability of glucose in the small intestine and the stimulation, at least in part, of the incretin GIP. The reduction in duodenal glucose and PPG in the face of lower insulin release but an increase in colonic fermentation in the rr genotype would appear to be solely due to an increase in starch reaching the colon.

The experiments in vitro and in vivo performed here demonstrated the complex multifactorial nature of the increased delivery of starch to the colon in the rr genotype. We observed that the cooked rr pea seeds appear more resistant to fracture and during simulated gastric and duodenal digestion the size of the particle population remained larger reducing the surface area for amylase activity as previously shown [27]. The metabolomic profiling of the aspirated gastric and duodenal samples indicated differences between the two genotypes in the amylopectin/maltotriose/maltose concentrations during digestion. It is known that amylopectin is more readily digested than amylose and that amylose is a poor substrate for pancreatic α-amylase [29]. By identifying higher concentrations of these metabolites in the digesta from the RR genotype it would suggest that the greater fracturing of the food matrix in the RR genotype leads to increases in digestible carbohydrate in the duodenum. We demonstrated that the complex nature of starch digestion and the size, morphology and physical chemistry of starch granules (helix ordering and chain length) are more accurate predictors of glycaemic response than simply the amylose content of the pea seeds. For example, we demonstrated that cooking rr pea seeds increased amylose double helix starch structure, creating resistant starch that is not seen in RR. This process has previously been demonstrated to increase resistance to amylase [19]. Our data also suggest that the penetration of α-amylase into rr cells is lower and slower than in RR not only in the samples digested in vitro but in duodenal samples from humans in vivo, similar to observations made in ileostomy volunteers using wheat flour and particles [25]. The studies in vitro clearly align with the stable isotope experimental studies in vivo which demonstrate a reduced absorption of carbohydrate in the small intestine with an increase in bacterial fermentation in the rr compared with the RR group, as judged by fractional recovery in 24 h urinary ¹³C SCFA propionate and butyrate profiles. SCFAs, particularly butyrate, are associated with numerous health benefits [30]. There was no detectable change in the stool 16S profile in the rr compared to the RR group at 24 h despite the increase in SCFA production. This suggests an increase in microbial carbon flux from the labelled carbohydrate in the colon from the rr peas without an acute change in microbial diversity compared to the RR peas. This effect was observed in both seeds and flour from the rr line highlighting the importance of the mutation on the starch compositional profile regardless of the food matrix, processing and preparation. These observations highlight the multicomponent aspect leading to reduced duodenal glucose.

Observations from the 28-day supplementation study, demonstrated no effect on glucose metabolism related biomarkers despite some positive changes in the gut microbiota, with an increase in the proportion of the genus Bifidobacterium following supplementation with the rr genotype. Studies have shown that Bifidobacterium abundance increase with enriched carbohydrate environment and has been associated with improvements and maintenance of metabolic health [31]. However, this concept was not proven here This outcome highlights that the impact of gut microbial metabolites and/or gut microbe/host immune interactions is possibly weakly associated with glucose metabolism improvements in healthy individuals.

It is important to highlight some limitations of this work. Our data focuses on acute differences in glycaemic responses from an overnight fasted state and, therefore, studies are needed to confirm these results in a real-life setting. The pea-derived products were single food products, which were added to the habitual diets of volunteers without any other alterations in their diets. Given the results from the acute studies, it might have been more efficacious to have a portfolio of products with an item eaten at each meal.

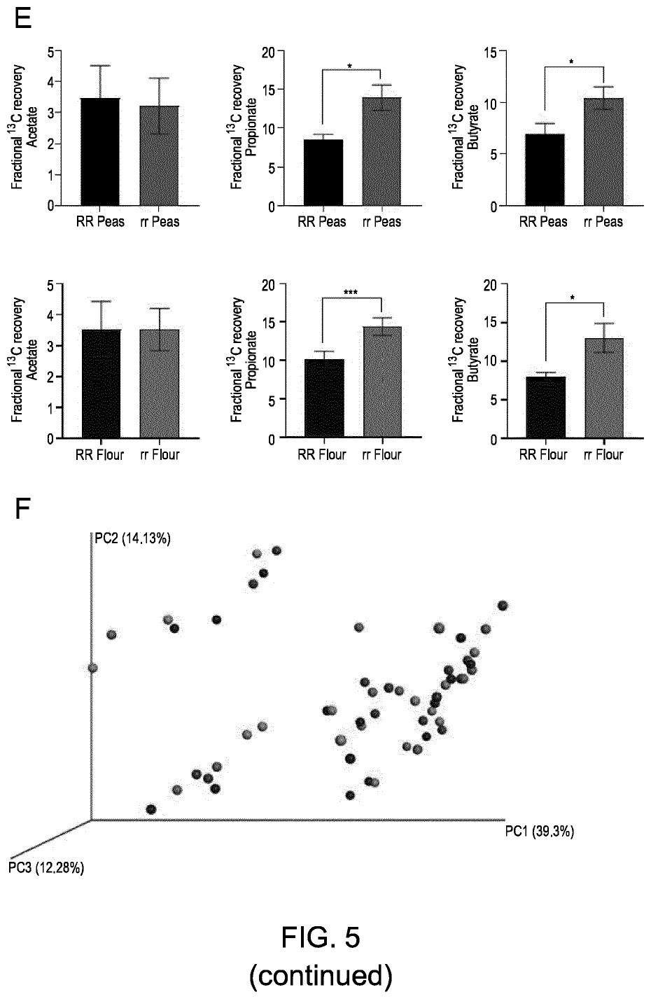

Our data shows that the impact of the contrasting pea genotypes on PPG it is due to complex differences in starch structure and food matrix and their impact on cooking and digestion. Additionally, to allow comparison with a common measure of carbohydrate quality we reported the glycaemic index value. This experiment confirms that rr pea seeds have a lower postprandial glucose response than RR, garden peas and glucose control (Supplementary FIG. 9).

These observations could be used to inform the production of modified food types, either through the selection of digestion resistant starch phenotypes or altered food matrices with an aim to drastically lower PPG, reducing risk of metabolic diseases at a population level.

Recent studies of SBE genetic mutations have extended to induced mutations in staple crops such as rice and wheat, adding potential and wide applicability for direct translation of our results. With modern genetic and genomic tools, the discovery or generation of sbe mutations across a number of seed and grain crops provides great potential for expansion of such food products to tackle major diseases, such as T2D. It is worth emphasizing that the naturally occurring mutation in pea, studied here, is one of the classical mutations studied by Mendel, on which the science of genetics is based. Accessions of pea carrying the sbe mutation have been commercially cultivated for several decades as a fresh vegetable crop. Introducing this mutation into pea crops for a broader range of food uses in underway, to provide ingredients for a wide range of industries.

### Example VI: Materials and Methods

### Food Materials

The food materials used during the experimental procedures are listed below:
1. Wild type pea seeds (BC1/19RR line), used as control group
2. Mutant pea seeds (BC1/19rr line), used as the treatment group
3. Wild type pea flour (BC1/19RR line), used as control group
4. Mutant pea flour (BC1/19rr line), used as treatment group
5. Wild type pea hummus and mushy peas (BC1/19RR line), used as control group
6. Mutant type pea hummus and mushy peas (BC1/19rr Line), used a treatment group.

The near-isogenic lines of pea (BC1/19RR, BC1/19rr) are available from the John Innes Centre Germplasm Resources Unit (JI3316=RR, round-seeded, JI3317=rr, wrinkled-seeded; https://www.seedstor.ac.uk). Bulked seed stocks were generated by growing plants on wire in field plots over successive seasons (March-July). The resulting seed stocks were used for studies in vivo and in vitro and supplied to the University of Glasgow for ¹³C labelling and Campden BRI to produce the pea derived products. Campden BRI developed the two pea products for the long-term study (trial 4).

¹³C labelling of pea seeds were sown in troughs in a glasshouse at the James Hutton Institute, Dundee. The plants developed well and were pulse labelled with ¹³CO₂ one week after flowering. The mature seed was collected and air dried. A sub-sample of each variety was milled to a fine flour and analysed for crude protein, C:N ratio, total ¹³C and starch ¹³C. The yield of the wild type was 1.24 kg at 0.242 atom % ¹³C excess, as measured by EA-IRMS at SUERC. The yield of the rr mutant was 1.26 kg at 0.133 atom % ¹³C excess.

### Human Clinical Trials

### Volunteers

Volunteers were provided with informed written consent forms prior to their participation in the 4 human clinical trial studies. We performed three acute studies (trial 1,2,3) and one randomized controlled trial (trial 4). In the acute studies 1 and 3 we recruited 10 volunteers per study as these were exploratory studies and the first time being conducted in humans. In the acute study 2, we recruited 12 volunteers. Although this was also an exploratory study, due to the nature and difficulty of the sample collection (intubation of gastric and duodenum of volunteers) we decided to include 12 volunteers to account for a possible higher dropout rate or difficulties in samples aspiration. This information was based on previous data by Steven Julious (2005) where he reported that a sample size between 10-12 is enough to gain precision in the mean and variance [32]. For the final study, which was a randomized cross over, double blind clinical trial data from Te Morenga et al. (2010), were used to estimate the required sample size. Assuming a mean±SD change in HOMA2-IR of 0.0±0.5 following the RR intervention and −0.3±0.5 following the rr intervention, a power calculation confirmed that 24 volunteers would be sufficient to detect a difference (α=0.05, power=0.80) [33]. All studies were approved by the South East Coast Surrey Research Ethics Committee (15/L0/0184) and carried out in accordance with the Declaration of Helsinki. Volunteers were recruited via a healthy volunteer's database and public advertisement. For the exploratory studies 1, 2 and 3 men and women aged 18-65 years old, with a body mass index (BMI) of 18.5-29.9 kg/m² were recruited. For the study 4, men and women 40 to 70 years old were recruited with same BMI as in studies 1, 2 and 3. Exclusion criteria included: weight gain or loss >3 kg in the previous 2 months, any chronic illness or gastrointestinal disorder, history of drug or alcohol abuse in the previous 2 years, use of antibiotics or medications likely to interfere with metabolic variable measured, smoking. All study visits took place at the National institute for Health Research/Wellcome Trust Imperial Clinical Research Facility, Hammersmith Hospital, London, United Kingdom and were conducted between May 2015 and December 2017. Randomization for all studies was generated by sealed envelope (Sealed Envelope Ltd, London, UK). In all human clinical studies volunteers were asked to consume the same standardized meal the evening before each study visit and avoid caffeine, alcohol consumption and strenuous exercise for 24 h before the experimental procedure. They were advised not to start any other new diets or intensive exercise regimes during the study period. Weight, height and body fat measurements were collected by using bioimpedance analysis (BC-418 Analyzer; Tanita UK).

### Trial 1—Study Day Protocol

This study was a randomized, controlled, double blind, cross-over trial. 10 volunteers were recruited for the study and attended 4 study visits (≥7 days apart) after an overnight fast. The study lasted for 300 minutes. Volunteers received a standardized test meal (0 min) (details of the test meal composition, Supplementary Table 12) with 50 g dry weight of RR or rr pea seeds and flour in a random order. The test meal included 100 mg ¹³C-octanoic acid (Sercon Ltd, Crewe, UK) which was injected in the yolk. ¹³C-octanoic acid breath test is a non-invasive, reproducible, stable isotope method for measuring solid phase gastric emptying. By measuring the level of ¹³CO₂ that appears in breath samples following oxidation of the absorbed tracer, we were able to calculate how quickly the stomach empties after eating. Breath samples were taken every 15 min until the end of the study day (300 min). The breath test poses no risk to the volunteers and involves blowing through a straw into an Exetainer (Labco Co., High Wycombe, UK) until vapour condensed at the bottom of the tube. Analysis of breath ¹³CO₂ enrichment was by continuous flow isotope ratio MS (AP2003, UK).

### Trial 2—Study Day Protocol

Twelve healthy volunteers were recruited for this randomized, controlled, double blind, cross over study. Volunteers attended the Clinical Research Facility for 4 consecutive days (3 nights). Nasogastric and nasoduodenal feeding tubes were placed to allow aspiration of samples from the stomach and small intestine. The enteral feeding tubes were placed by a doctor using the CORPAK (MedSystems, Halyard UK) feeding tube model that tracks the position of the tube during placement without the need for x-rays. The tubes remained in place for the duration of the 4-day visit. Each visit lasted for 180 min. An intravenous cannula was inserted into one arm for blood sampling of plasma, serum and gut hormones. Each morning, fasting blood samples and gastric content samples were taken at −10 and 0 min. In random order, volunteers received at 0 min, a portion of 50 g dry weight RR or rr pea seeds and/or flour. Postprandial blood samples were collected at 15, 30, 60, 90, 120, 180 min. The macronutrient profile of pea seeds can be found in the Supplementary Table 13.

### Trial 3—Study Day Protocol

Ten healthy volunteers were recruited for this randomized, controlled, double blind, cross over study and attended the research facility 4 times (≥7 days apart) after an overnight fast. Each study visit lasted for 8 h. Volunteers received a test meal (0 min) which contained 50 g dry weight ¹³C pea seeds or flour in random order. Details of the composition of the test meal are in Supplementary Table 14. Throughout the study volunteers collected urine samples and were advised to keep collecting their urine samples until the following morning (24 h). The following morning, they returned to the research unit with the urine sample and a stool sample.

### Trial 4—Study Day Protocol

Twenty-five volunteers were recruited for this randomized, controlled, double blind, cross over study and attended the research facility for 4 visits. Volunteers had to undergo two separate 28-day supplementation periods and they were provided with mushy peas and pea hummus products (RR or rr line). They were advised to consume 1 can of each product per day. The products were matched for dry weight. Each can contained 50 g dry weight of peas in a total amount of 210 g. Supplementary Table 4 presents the macronutrient profile of RR and rr mushy peas and pea hummus analysed by Campden BRI. Before and at the end of each 28-day supplementation period they attended the research facility for a study visit. At time 0 volunteers received an ENSURE drink (Ensure Vanilla Nutrition Shake, Abbott; 330 ml, 66.6 g carbs, 20.5 g protein and 16.2 g fat) consisting of 500 kcal. Blood samples were collected throughout the time course of the study (5 h). Urine samples were collected for the same time frame. Volunteers had to collect a stool sample the day before each study visit. There was a 28-day washout period between the two supplementation periods.

### ¹³C Breath Sample Analysis

Breath samples analysis was performed by isotope ratio mass spectrometry (IRMS) [34]. Breath samples were collected by exhalation of expired breath into an Exetainer (Labco Ltd, Lampeter, Ceredigion, United Kingdom) using a straw. Volunteers were encouraged to continue to blow into the Exetainer until condensate was observed in the base of the tube indicating alveolar breath collection [35]. Collected breath samples were analysed by flushing a portion of breath with helium gas into the IRMS where water is removed, and CO₂ separated from other gas species using gas chromatography before introduction into the mass spectrometer (AP2003, GV Instruments, Manchester, UK). The isotope ratio ¹³C:¹²C was calculated from the ion abundance of m/z 44, 45 and 46 with reference to a laboratory reference CO₂ (itself calibrated against Vienne Pee Dee Belemnite (VPDB)) with correction of the small contribution of ¹²C¹⁶O¹⁷O at m/z 45, the Craig correction. Breath δ¹³C enrichment (‰) over baseline was calculated for each timepoint and the envelope of breath ¹³C excretion was analysed using a modified version of the curve-fitting techniques to compute gastric emptying T_{1/2} times [17].

### Biological Sample Collection and Processing

Ten millilitres of blood were collected at each timepoint for assay of plasma glucose (EDTA), serum insulin, and plasma gut hormones (3 ml in lithium heparin tube containing 60 μl aprotinin protease inhibitor; Nordic Pharma UK). All blood sample tubes were centrifuged at 2500 g for 10 min at 4ºC. Samples were separated and frozen at −80ºC until the end of the study when analysis took place.

### Biological Sample Analysis

Plasma glucose and luminal glucose analysis was performed using Randox Glucose (GLU/PAP) kit supplied by Randox using 20 μl of plasma glucose. A human insulin radioimmunoassay kit (Millipore) was used for analysis of insulin based on manufacturer's specification with 50 μl serum. GLP-1 was measured with the use of previously established in-house specific and sensitive radioimmunoassay. GIP was measured by using an ELIZA Human GIP (Millipore) based on manufacturer's specification with the use of 20 μl serum sample.

### Experiments Ex Vivo

FITC labelled α-amylase was added to a suspension containing pea cells. Images of the cells were taken at different time points using an Olympus BX 60 Fluorescence Microscope or a Zeiss LSM 880 Confocal Laser Scanning Microscope.

### Metabolomic Gastric and Duodenal Samples Analysis

We assessed the gastric and small intestinal metabolic profiles of the aspirated samples using the metabolic profiling approach. Each metabolic profile contains hundreds of metabolites measured in an untargeted manner by Proton Nuclear Magnetic Resonance (¹H-NMR) spectroscopy.

### Samples Extraction

Gastric and duodenal samples were centrifuged for 15 min at 3000 g. Metabolites were extracted from the gastric and duodenal samples using a modified Folch extraction procedure. Two millilitres of chloroform/methanol (2:1) ratio where added to 450 μl of each gastric and duodenal sample. This mixture was vortexed, and 1 ml of purified water was added. Samples were vortexed for 1 min and centrifuged for another 20 min at 3000 g, at 0° C. This method produced two phases and metabolites split into the aqueous and the organic phase according to their polarity. The aqueous phases were separated and evaporated to dryness using a speed vacuum concentrator and the dried sample was stored at −80ºC prior to analysis. The NMR profiles of the stomach and duodenal digested samples were analyzed by ¹H high resolution NMR spectroscopy.

### Sample Treatment

The dried aqueous phase of the gastric samples was re-constituted in 540 μl of H₂O and sonicated for 20 min. 540 μl were mixed with 60 μl of a 3M phosphate buffer (pH 7.4, 80% D20) containing 1 mM of the internal standard, 3-(trimethylsilyl)-[2,2,3,3,2H4]-propionic acid (TSP) and the mixture transferred to the 5 mm NMR tubes. The dried aqueous phase of the duodenal samples was re-constituted in 640 μl of H₂O and sonicated for 20 min. 540 μl was mixed with 60 μl of a 1.5M phosphate buffer (pH 7.4, 80% D₂O) containing 1 mM of TSP and the mixture was transferred to 5 mm NMR tubes. Quality control samples were prepared independently for gastric and duodenal samples by pooling 90 μl of each sample.

NMR metabolite identification strategies were used as described by Garcia-Perez et al [36].

### Quantification of ¹³C Plasma Glucose

Plasma samples were diluted 1:5 with L-fucose internal standard. The ¹³C natural abundance of L-fucose was separately calibrated against VPDB and used as a chemical and isotopic internal standard. 0.5 ml of plasma was diluted with 2 ml internal standard. Samples then underwent ultrafiltration using 30000 molecular weight cut-off ultrafiltration devices (Amicron Ultra 4; Millipore, Watford, UK) at 3600×g for 45 min to remove proteins and other high molecular weight compounds. After this step, the samples were stored in two separate aliquots at −20° C. for further analysis. Analysis by liquid chromatography-IRMS (LC-IRMS) was performed as previously described. Fucose and glucose peak areas and background-corrected isotope ratios were exported to a spreadsheet for analysis. Glucose enrichment (o ¹³C (‰) was calculated using an in-house routine and using a relative ratio analysis approach against the IS for each sample to report the enrichment of glucose relative to VPDB and glucose ¹³C concentration, as the product of enrichment×concentration at each time point. Glucose concentration was calculated from the area ratio of the glucose peak area relative to fucose.

### Quantification of ¹³C SCFAs in Urine Samples

Samples were analysed as previously described modified to increase sensitivity of the analysis. In brief, urine samples (7 ml) were spiked with 200 nmoles 3-methyl valerate (3 mV; internal standard) and 200 L NaOH (300 mmoles/L). A ‘process blank’ was prepared containing freshly deionized water and identical spikes of 3 mV and NaOH. Samples and blanks for each run were dried on a vacuum concentrator (Jouan RC10 Vacuum Centrifuge, ThermoFisher, Paisley, UK) at ambient temperature. Dried samples were acidified with 100 μl HCl and SCFA extracted with 400 μl methyl-tert butyl ether. 300 μl of the MTBE phase was removed to clean vials for analysis by GC-C-IRMS as previously described [37]. The isotopic enrichment of each SCFA was calculated relative to 3 mV which itself had been calibrated against laboratory standards and VPDB. Enrichment of each SCFA with time was expressed relative to the enrichment of the starting pea material ingested to derive a fractional ¹³C enrichment curve for each SCFA.

### Bacterial Composition Analysis of Stool Samples Collected for Study 3

Total DNA was extracted from stool samples (˜200 mg) using the FastDNA SPIN Kit for Soil (MP Biomedicals, UK) with a bead-beating step (Kellingray et al., 2017). DNA yield was quantified using the Qubit fluorometer prior to the samples being sent to the Earlham Institute (Lindström et al.), where the V4 hypervariable regions of the 16S rRNA genes were amplified using the 515F and 806R primers with built-in degeneracy (Caporaso et al., 2011). The amplicons were sequenced using paired-end Illumina sequencing (2×250 bp) on the MiSeq platform (Illumina, USA). Sequencing data were analysed using the Quantitative Insights Into Microbial Ecology (QIIME) 1.9 software and RDP classifier 16S rRNA gene sequence database (Wang et al., 2007). The trimmed reads were filtered for chimeric sequences using ChimeraSlayer, bacterial taxonomy assignment with a confidence value threshold of 50% was performed with the RDP classifier (version 2.10), and trimmed reads clustered into operational taxonomic units at 97% identity level. Weighted and unweighted UniFrac distances were used to generate beta diversity principal coordinates analysis plots, which were visualised using the Emperor tool.

### Bacterial Composition Analysis of Stool Samples Collected for Study 4

Stool samples were collected at baseline and follow up at each supplementation period. The samples were stored at −80ºC for between 6-9 months before processing. DNA was extracted from approximately 250 mg of stool samples using the PowerLyzer PowerSoil DNA Isolation Kit (Mo Bio, Carlsbad, CA, USA) following the manufacturer's instructions. Samples were bead beaten for 3 min at speed 8 in a Bullet Blender Storm (Chembio Ltd, St. Albans, UK) and this was the only modification to the protocol. All samples were analysed in a single batch. Sample libraries were prepared by amplifying the V1-V2 region of the 16S rRNA gene following Illumina's 16S Metagenomic Sequencing Library Preparation Protocol with the following alterations. First, the index PCR reactions were cleaned up and normalised using the SequalPrep Normalization Plate Kit (Life Technologies, Paisley, UK). In addition, sample libraries were quantified using the NEBNext Library Quant Kit for Illumina (New England Biolabs, Hitchin, UK). Sequencing was performed on an Illumina MiSeq platform (Illumina Inc., Saffron Walden, UK) using the MiSeq Reagent Kit v3 (Illumina) using paired-end 300 bp chemistry. The resulting sequencing data was processed following the DADA2 pipeline as previously described. The SILVA bacterial database version 132 was used to classify the sequence variants. The UniFrac weighted distance matrix generated from Mothur was used to generate non-metric multidimensional scaling (NMDS) plots and PERMANOVA p-values using the Vegan library within R (Dessau and Pipper, 2008). Due to high inter-individual variability we examined the data as paired samples per volunteer. Differences in microbial communities between and within groups were tested by using the Wilcoxon signed-rank test.

### Digestion In Vitro—Study Design

### Pea Preparation

Pea seeds were milled by electric coffee grinder (Krups, Berkshire, UK), and were sieved to 1 mm particles (Cole-Palmer, St. Neots, UK). All chemicals, reagents and enzymes were supplied by Sigma Aldrich (Dorset, UK). Approximately 5 g pea seeds were soaked overnight in 100 mL ultrapure water (room temperature). Flour (1 g) was weighed into 15 mL Pyrex tubes (screw cap with PTFE cap liner) and mixed with ultrapure water (4:1). Samples were hydrated, 1 h at room temperature and cooked (1 h, in a boiling water bath), cooled and further diluted (8:1). Peas were boiled for 1 h in ultrapure water, drained, and skins were removed from both uncooked and cooked peas. To mimic chewing, peas were pushed through a garlic press (Lakeland, UK) to produce chunks with particle sizes ≥2.5 mm.

### Simulated Digestion

Triplicate digestions of flours and pea chunks were performed using a standardised static biochemical model developed by Minekus et al [38], with modifications to the composition of the simulated digestion fluids. In all cases sodium bicarbonate and ammonium bicarbonate were directly substituted with bis-tris, due to its buffering capacity which was important for maintaining pH 7.0 in the intestinal phase. Oral phase: simulated salivary fluid (SSF) [15.1 mM KCl, 3.7 mM KH₂PO₄, 13.66 mM bis-tris, 0.15 mM MgCl₂(H₂O)₆, 1.5 mM CaCl₂)(H₂O)₂] was added, 1:1 v/w, to samples immediately followed by human salivary amylase (product code A1031: type XIII-A lyophilised powder—α-amylase from human saliva) providing a final concentration of 75 U/mL, then incubated for 2 min at 37° C.

Gastric phase: at 2 min, the pH was adjusted to 3.0 (+0.05) using 0.1M HCl, simulated gastric fluid (SGF) [6.9 mmol KCl, 0.9 mmol KH₂PO₄, 25.5 mmol bis-tris, 47.2 mmol NaCl, 0.1 mmol MgCl₂(H₂O)₆, 0.15 mmol CaCl₂)(H₂O)₂] was added (1:1 v/v). Finally, pepsin (product code P7012: pepsin from porcine gastric mucosa) was added providing a final concentration of 2000 U/mL. The gastric phase was incubated at 37° ° C. for 1 h. The recommended time for gastric digestion is 2 h however, based on the lack of starch degrading enzymes in the gastric phase, the time for these experiments was reduced.

Intestinal phase: immediately after the gastric phase the pH was raised to 7.0 (±0.05) using 0.1M NaOH, simulated intestinal fluid (SIF) was added [6.8 mM KCl, 0.8 mM KH₂PO₄, 85 mM bis-tris, 38.4 mM NaCl, 0.33 mM MgCl₂(H₂O)₆, 0.6 mM CaCl₂)(H₂O)₂, and 10 mM bile] (1:1 v/v) and finally pancreatin (product code P7545: pancreatin from porcine pancreas) was added providing a final concentration of 100 U/mL. The intestinal phase was incubated at 37° C. 170 rpm) for 2 h.

Flour was digested in a heated mixing vessel where samples were stirred continuously (500 rpm) at 37° C. The pH of the intestinal phase was maintained at 7.0 by KEM AT-700 automatic titrator (Kyoto Electronics, Leeds, UK). At the end of each phase of digestion, 0.1 mL samples were taken: oral phase 2 min; gastric phase 60 min, intestinal phase 120 min.

Pea chunks were digested in disposable centrifuge tubes (Greiner Bio-One Ltd, Stonehouse, UK) at 37° C. in an orbital shaking incubator (Sartorius, Goettingen, Germany) at 170 rpm, and sample collection times were the same as for the flour.

### Starch Assay

Uncooked and cooked pea chunks (100 mg±5 mg) were digested according to protocol described in section simulated digestion. The liquid phase was removed from the samples by centrifugation (2000 g for 5 min). Additional digested samples were homogenised at 1000 rpm, using a T25 Ultra-Turrax (IKA, Oxford, England), post-intestinal digestion phase, to check that all starch in the pea chunks had been accounted for by the assay. After milling, samples were centrifuged at 10000 g for 10 min, and the pellet was retained.

Total and resistant starch contents of undigested and digested flours and pea chunks were determined using assay kits purchased from Megazyme International (Co. Wicklow, Ireland).

Total starch (assay procedure K-TSTA 07/11). Samples were heated in aqueous ethanol (80% v/v) at 80-85° C. for 5 min and centrifuged at 1800 g for 10 min. Supernatants were decanted, and excess liquid was drained from the pellets.

Resistant starch (assay procedure: KRSTAR 09/14). Samples were incubated with 4.0 mL pancreatic α-amylase (30 U/mL) and AMG (3 U/mL) for 16 h at 37° ° C. with continuous shaking (200 rpm), during which time non-resistant starch was solubilised and hydrolysed to D-glucose. Enzymes were halted by washing with 4.0 mL ethanol (99% v/v), followed by centrifugation at 1500 g for 10 min. Supernatants were decanted and pellets were re-suspended in 8.0 mL 50% ethanol, the centrifugation step was repeated, and followed by a final washing step. Supernatants were decanted, and excess liquid was drained from the pellets.

All pellets were incubated in 2.0 mL 2 M KOH for 20 min on ice and neutralised in 8.0 mL 1.2 M sodium acetate buffer (pH 3.8). Starch was hydrolysed to form maltodextrins by addition of thermostable α-amylase to give a final content of 3.0 U/mL. The maltodextrins were further hydrolysed by addition of AMG to give a final content of 3.3 U/mL, to form D-glucose.

Total starch and resistant starch contents were determined by incubating 0.1 mL of hydrolysed samples with 3.0 mL GOPOD reagent [glucose oxidase plus peroxidase and 4-aminoantipyrine in reagent buffer (4-hydroxybenzoic acid) at 50° C. for 20 min, where the D-glucose was oxidised to D-gluconate, which was quantitively measured in a colorimetric reaction. The absorbance for each sample and D-glucose controls was read at 510 nm against the reagent blank using UV tolerant cuvettes (Sarstedt Limited, Leicester, UK) and a Lambda UV/Vis spectrophotometer (Perkin-Elmer, Buckinghamshire, UK).

### Starch Structural Analysis—SEC and ¹³C CP/MAS NMR

SEC analysis was conducted on debranched, purified starch samples using a Waters Advanced Polymer Characterisation System as described in [39].

Solid-state ¹³C CP/MAS NMR experiments on all pea and flour powder samples were carried out on a Bruker Avance III 300 MHz spectrometer, equipped with an HXY 4-mm probe, spun at a frequency of 12 kHz, at a ¹³C frequency of 75.47 MHz, and MAS of 54.7°. Samples were manually ground using a mortar and pestle and approximately 110-130 mg of each sample was packed into a 4-mm cylindrical partially-stabilised zirconium oxide (PSZ) rotor with a Kel-F end cap. The ¹³C CP-MAS NMR experimental acquisition and processing parameters were 90° 1H rf pulse width of 3.50 us and 90° 13C rf pulse width of 4.50 μs, contact time of 1000 μs, recycle delay of 5 s, spectral width of 22.7 kHz (301.1 ppm), acquisition time of 28.16 ms, time domain points (i.e. size of FID) of 1280, line broadening was set to 20, 6144 number of scans and 16 dummy scans. All experiments were referenced to tetramethylsilane and hexamethylbenzene for 1H and 13C, respectively, and carried out at approximately 26° C.

Calculation of starch molecular (double helical) order was performed following the procedure described by Flanagan et al [40]. In brief, following determination of the free induction decay of all samples, the data was Fourier transformed, phase corrected and zero-filled to 4096 data points. Chemical shift vs relative intensity data was used to obtain an estimation of the total crystallinity of each sample analysed using partial least squares analysis against a reference set of 114 spectra of starch with known values of molecular order obtained using spectral deconvolution and referenced against x-ray diffraction data.

### Particle Size

Pea chunk size (cooked) was determined after gastric and intestinal simulated digestion by dynamic light scattering (DLS), using an LS13320 laser diffraction particle size analyser (Beckman-Coulter, Buckinghamshire, UK), and using starch as the optical model with PIDS (Polarization Intensity Differential Scattering) obscuration ≥45%. The mean particle size distribution was measured 3 times over 60 second intervals.

### Compression Test Experiments

An Instron machine 5540 was used to conduct the test with a 10 N load cell, model 2530-428, and was connected to Bluehill3 software for the collection and analysis of the results.

RR and rr pea seeds were tested, using the same cooking method as for trial 2. Ten seeds from each pea line using three different batches were measured for length and height using digital Vernier caliper to ensure similar geometry between peas. To conduct a test, a sample seed was placed in the most stable position prior to testing. A flat plate attached to Instron was used to apply load to the seed. The compression test was performed at speeds of 1 mm/s and 15 mm/s. The force versus deformation curves were obtained until rupture of the seed occurred.

### Microscopy

Microscopy was used to characterise the pea seeds and flour throughout the digestion process and to visualise any changes to the macro and microstructure of the foods. It was particularly important to image areas of damaged tissue from the action of chewing, as these areas were accessible to enzymes and therefore would be susceptible to digestion.

### Light Microscopy

Light microscopy was used to characterise the macro and microstructure of pea seeds and flour. Uncooked flour samples were hydrated in ultrapure water 20 min before imaging; cooked and digested flour samples were imaged immediately after cooking and digestion steps. Iodine (0.2% iodine in 2% potassium iodide, aqueous) was used to stain starch.

Uncooked, cooked and digested pea chunks of approximately 1 mm³ were fixed in 2.5% glutaraldehyde/2% formaldehyde in 0.1M PIPES buffer for 8 days, to improve starch polymerisation, using 2.5% glutaraldehyde alone was not adequate for RR starch. The pea chunks were washed 3 times in 0.1M PIPES buffer for 15 min each. The chunks were then post-fixed in 1% osmium tetroxide (aqueous) for 2.5 h before 3×15-min ultrapure water washes and an ethanol series dehydration (10, 20, 30, 40, 50,60, 70, 80, 90, 100%) with at least 15 min between ethanol changes. The final ethanol change was repeated twice more with 100% ethanol. The last ethanol wash was replaced with a 1:1 mix of LR White medium grade resin (London Resin Company Ltd) to 100% ethanol and put on a rotator for 1 h. This was followed by a 2:1 and a 3:1 mix of LR White resin to 100% ethanol and finally 100% resin, with at least 1 h on the rotator between each change. After 1 h in 100%, the resin was changed twice more with fresh 100% resin with periods of at least 8 h on the rotator between changes. Four blocks from each sample were each put into BEEM capsules with fresh resin and polymerised overnight at 60° C. Semi-thin sections approximately 1 μm thick were cut using an ultramicrotome (Ultracut E, Reichert-Jung) with a glass knife mounted with an ultrapure water-filled trough. The sections were picked up and transferred onto a drop of water on a glass slide and dried in an oven at 100° C. The sections were then stained with toluidine blue (1% toluidine blue in 1% sodium borate, aqueous) for protein and iodine (0.2% iodine in 2% potassium iodide, aqueous) for starch for only a few seconds and then rinsed with water before being dried again in the oven. The slides were then ready to view under the microscope (Olympus BX60 microscope).

### Scanning Electron Microscopy (SEM)

Pea chunks were fixed using a 2.5% glutaraldehyde/0.1M PIPES buffer (pH 7.4) for 5 days. After washing with 0.1M PIPES buffer, the chunks were dehydrated in a series of ethanol solutions (10, 20, 30, 40, 50, 60, 70, 80, 90, 3×100%) and 3×100% ethanol. Samples were critical point dried in a Leica EM CPD300 critical point drier using liquid carbon dioxide as the transition fluid and mounted onto SEM stubs with silver paint (Agar Scientific, Stansted, UK). The samples were coated with gold in an Agar high resolution sputter-coater apparatus. Scanning electron microscopy was carried out using a Zeiss Supra 55 VP FEG SEM, operating at 3 kV.

### Statistical Analysis

Data were analysed using Graph Pad Prism (GraphPad Software, San Diego, CA, USA), IBM SPSS (Statistics for Windows, Version 24, Armonk, NY, USA) or MatLab version R2014a, The Mathworks, Inc.; Natwick, MA). Data were tested for normality using Shapiro-Wilk Test. Comparison of time series data was carried out by two-way analysis of variance (ANOVA) with post hoc LSD Fisher correction. Areas under the curve (AUC) were calculated using the trapezoidal rule and were compared using paired Student's t-test. AUCs were calculated based on the time frame and parameters of each study. AUC_{0-120 min} was calculated as this is a dynamic representation of the meal effect on postprandially glycaemia which was the primary aim of this experimental study (study 2). In study 3, AUC_{0-480 min} was used as ¹³C labelled pea seeds and flour were used. The scope of this study was to understand the whole-time curve and not driven by conclusion about the test meal per se. As peas and flour were labelled with ¹³C this allowed us to trace fuel metabolism and therefore a time frame to capture both digestion and fermentation data was used. All results and graphs are expressed as mean±SEM. Results were considered statistically significant when p<0.05, two sided with the significance level indicated as *p<0.05, **p<0.01, ***p<0.001.

SEQUENCE LISTING

SEQ ID NO: 1: P.sativum SBE1 cDNA
ccattcttca ccgtacaata catccctatc tataataaac aacaattttc ttctcatcat
tatatcattt tattcaatta ttaaatagtt ttattctttc aagaagttaa tgcgaatcat
tttttactct gtatgtcatc acacttctct tgcctccttc tactcaattt tttagatctc
ttcttctttg caatataaac cctaagctga tagaagaaga tggtttatac aatctcagga
attcgatttc ccgttcttcc ttctctgcac aagtcgacct tacgctgcga tcgcagagct
tcatctcact ctttttttct caagaacaac tcgtcatcat tctctcggac ctcactttat
gcaaagtttt cccgcgattc tgaaaccaaa tcttccacaa ttgctgaatc tgataaagta
cttattcctg aagatcaaga taactctgta tccttggcag atcaacttga aaatcctgat
ataacctcag aggatgcaca gaacttggag gatttaacca tgaaagatgg gaataagtac
aatattgatg aatcaactag cagttataga gaggttggag atgagaaagg ttctgttacg
tcatcatcac ttgtagatgt taacaccgac actcaagcca agaagacatc agttcattca
gacaagaaag taaaagtaga taaacctaag atcattcctc cacccggtac tgggcagaaa
atatatgaga ttgatccact tttgcaagct caccgtcagc atcttgattt ccgttatgga
caatacaaaa gaattcggga ggaaattgac aaatatgaag gtggtcttga tgcattttcg
cgtgggtacg aaaagtttgg cttcacacgc agtgctacag gcattactta cagggagtgg
gcacctggag ctaagtcagc agcattagtt ggagatttca acaattggaa tccaaatgca
gatgtaatga ctaaggacgc ttttggtgta tgggagatct tcttgccaaa caatgccgat
ggttcgccac caattcctca tggttctcga gtcaagatcc acatggatac tccctctggg
atcaaggact cgattcctgc ttggatcaaa ttctctgtac aggctcctgg tgaaattcca
tacaatggaa tatactatga tcccccagag gaggaaaagt atgtcttcaa acatccacag
ccaaaacgac cacagtcaat tagaatatat gagtcacaca ttggaatgag tagcccggag
ccaaaaatca acacatatgc gaactttaga gatgatgtac tacctcgcat taaaaaactt
ggctataatg ctgtccagat tatggctatc caagaacatt cttattatgc tagttttggg
taccatgtta ctaatttctt tgcacctagc agtcggtttg gtactccaga agatctcaag
tctctgatag acagagccca tgaactaggc ctgcttgttc tgatggatat tgtacatagc
cattcctcaa ataatacatt ggatggcctg aacatgtttg atggaactga tggtcattac
ttccatcctg gttcacgggg ttatcattgg atgtgggatt ctcgcctttt taactatgga
agctgggaag tactaaggta cctactttca aatgcaagat ggtggctgga tgaatataag
tttgatgggt ttcgatttga tggcgtcaca tcaatgatgt acactcatca tggactgcag
gtatctttta ccggaaatta cagtgagtat tttggtttag caactgatgt tgaggctgtg
gtttacatga tgcttgttaa tgatctcatt cacgggctct tccctgaagc tgtttcaatt
ggtgaagatg tgagtggaat gccaacattc tgccttccga cgcaagatgg tgggattggc
tttaactacc gcttgcatat ggctgttgca gacaagtgga ttgagcttct taagaaacaa
gatgaagatt ggagaatggg tgatatagtc cacacactga caaacagaag gtggctggaa
aaatgtgtgg tttatgctga aagtcatgac caggccttgg ttggtgataa gacactagca
ttttggttga tggacaagga tatgtatgac ttcatggctt tagacaggcc atctactcct
ctaatagatc gtggtatagc attacacaaa atgattcggc ttattactat gggccttggt
ggtgaagggt atttgaattt tatggggaat gaattcggac atcctgagtg gatcgatttt
ccaaggggtg agcaacatct tcctaatggc aaaatagttc ctgggaataa caacagttat
gataaatgca gacgtagatt tgacttgggt gatgcagatt atctaagata tcatggaatg
caagaatttg atcgagctat gcagcatcta gaagagagat atggtttcat gacttctgag
caccaatata tttcacggaa aaatgaaggt gacagagtta taatctttga aagagacaac
ctggtctttg tcttcaattt tcactggacc aacagttatt cagattacaa ggttggctgt
ttaaagccag ggaaatataa gattgtcttg gattcagatg ataccttgtt tggtggcttc
aataggctca atcatactgc tgagtacttc acctctgaag ggtggtatga tgaccggcct
cgatctttcc tcgtttatgc accttctaga acagcagtgg tgtatgccct tgcggatgga
gttgaatcag agccaattga actgtcagat ggagtagaat cagagccaat tgagctgtca
gttggagttg aatcagagcc aattgagctg tcagttgaag aagctgaatc agagccaatt
gagcggtcag ttgaagaagt tgaatcagag acaactcaac agtcagttga agttgaatca
gagacaactc aacagtcagt tgaagttgaa tcagagacaa ctcaatagtc agttgaagtt
gaaccatgat gacaagtttt tctggaatga atatatcaat ttcataatgg catcagtgca
atcaagtcaa aaccagccaa ccaacttaag gtgattagag caggctctga aactgcaagg
cggagccctt gtgttaactt aatctttagt gcggcagcaa accaacgttg aagtgtttgt
acccattgct tgctaggtca attatgtata gcacgtcaag ttttttgttt ttacttgggg
tttaataggc acaagttgcg tggcgtgtga atgtgaaaca acaaatgtac tgtcctcaga
tcctccgttt ctgtggacat ataaatcctt tttacccata agtgcacaaa ccttaattaa
accctggaaa gttttgcggg aggccttata tatgatttct cctttcagaa ttaaaagttg
agtcatagca atagggccgt gctgatgaaa taattgtttt gtacacggtg aaaatagtat
tgccttacta taaaatataa atggtggaac ctttgcctcc aattgcaaaa aaaaaaaaaa
aaggaattc
SEQ ID NO: 2: P.sativum SBE1 amino acid
MVYTISGIRFPVLPSLHKSTLRCDRRASSHSFFLKNNSSSFSRTSLYAKFSRDSETKSST
IAESDKVLIPEDQDNSVSLADQLENPDITSEDAQNLEDLTMKDGNKYNIDESTSSYRE
VGDEKGSVTSSSLVDVNTDTQAKKTSVHSDKKVKVDKPKIIPPPGTGQKIYEIDPLLQ
AHRQHLDFRYGQYKRIREEIDKYEGGLDAFSRGYEKFGFTRS
ATGITYREWAPGAKSAALVGDENNWNPNADVMTKDAFGVWEIFLPNNADGSPPIPH
GSRVKIHMDTPSGIKDSIPAWIKFSVQAPGEIPYNGIYYDPPEEEKYVFKHPQPKRPQS
IRIYESHIGMSSPEPKINTYANFRDDVLPRIKKLGYNAVQIMAIQEHSYYASFGYHVT
NFFAPSSRFGTPEDLKSLIDRAHELGLLVLMDIVHSHSSNNTLDGLNMFDGTDGHYF
HPGSRGYHWMWDSRLFNYGSWEVLRYLLSNARWWLDEYKFDGFRFDGVTSMMY
THHGLQVSFTGNYSEYFGLATDVEAVVYMMLVNDLIHGLFPEAVSIGEDVSGMPTF
CLPTQDGGIGFNYRLHMAVADKWIELLKKQDEDWRMGDIVHTLTNRRWLEKCVVY
AESHDQALVGDKTLAFWLMDKDMYDFMALDRPSTPLIDRGIALHKMIRLITMGLGG
EGYLNFMGNEFGHPEWIDFPRGEQHLPNGKIVPGNNNSYDKCRRRFDLGDADYLRY
HGMQEFDRAMQHLEERYGFMTSEHQYISRKNEGDRVIIFERDNLVFVFNFHWTNSY
SDYKVGCLKPGKYKIVLDSDDTLFGGFNRLNHTAEYFTSEGWYDDRPRSFLVYAPS
RTAVVYALADGVESEPIELS
DGVESEPIELSVGVESEPIELSVEEAESEPIERSVEEVESETTQQSVEVESETTQQSV
EVESETTQ

### Tables

TABLE S1
Demographic characteristics of volunteers recruited and completed Study 1¹

| Characteristics | | | | | | |
|---|---|---|---|---|---|---|
| All (n = 10) | Screening | Visit 1 | Visit 2 | Visit 3 | Visit 4 | P Value |
| Gender (M:F) | 3:7 | | | | | |
| Age (years) | 49.3 ± 8.5 | | | | | |
| Weight (kg) | 68.0 ± 4.1 | 67.3 ± 4.1 | 67.5 ± 4.2 | 68.0 ± 4.1 | 68.0 ± 3.9 | 0.26 |
| BMI (kg/m²) | 24.0 ± 0.2 | 23.8 ± 0.1 | 23.9 ± 0.1 | 24.3 ± 0.1 | 24.2 ± 0.2 | 0.38 |
| Body Fat (%) | 27.4 ± 7.1 | 27.4 ± 1.7 | 27.7 ± 2.0 | 27.5 ± 1.6 | 27.6 ± 1.6 | 0.44 |

¹Results presented as mean ± SEM

TABLE S2
Demographic characteristics of volunteers
recruited and completed Study 2¹

| Characteristics All (n = 12) | Mean of 4 Consecutive Visits |
|---|---|
| Gender (M:F) | 7:5 |
| Age (years) | 45.8 ± 4.4 |
| Weight (kg) | 69.5 ± 1.9 |
| BMI (kg/m²) | 24.4 ± 0.6 |
| Body Fat (%) | 22.9 ± 2.4 |

¹Results presented as mean ± SEM

TABLE S3
Amylopectin/Maltotriose/Maltose found in
gastric samples associated with differences
found in the RM-MCCV-PLS-DA model between
the consumption of RR and rr peas¹
RR and rr Peas
Gastric Samples at 30 min

| | | | P-Value | Q-Value |
|---|---|---|---|---|
| | | | for | for |
| | Chemical Shift | Association | Peas | Peas |
| Metabolite | (multiplicity) | t = 30 | t = 30 | t = 30 |
| Amylopectin/ | 5.42(d), | ↑¹ | 0.000453 | 0.0024 |
| Maltotriose/ | 3.60(dd), | | | |
| Maltose | 3.57(dd), | | | |
| | 3.44(dd), | | | |
| | 3.29(dd) | | | |

¹Sign of association; ↑ Indicates higher excretion with RR, Multiplicity key is as follows: d—doublet, dd—doublet of doublets

TABLE S4
¹H and ¹³C NMR peak assignments for amylopectin, maltotriose, maltose

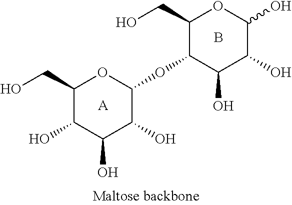

| | ¹H NMR: δ_{H} | ¹³C NMR: δ_{C} | Multiplicity and type |
|---|---|---|---|
| Name | (ppm) | (ppm) | of protons^{ª} |
| Amylopectin | | | |
| Maltose units with lineal 1→4 | 5.35-5.39 | 102.58 | 4d, CHs |
| linkage | 4.98 | 100.98 | d, CH |
| Branching unit with 1→6 linkage | 3.62^{C} | ~ | ~ |
| (C₁,H₁) | ~ | 75.98^{d} | ~ |
| Glucan moieties from branching at | 5.00-5.01 | 100.75 | 2d, CHs |
| position 6 (C₁,H₁) | 3.85^{c} | ~ | ~ |
| | ~ | 72.16^{d} | ~ |
| α/β-Maltotriose and α/β- | | | |
| Maltose^{b} | | | |
| Ring A | | | |
| | 3.42-3.46 | 72.16 | 3dd, CHs |
| | 3.59 | 74.36 | dd, CH |
| | 3.60 | 74.46 | dd, CH |
| | 3.69 | 75.74 | dd, CH |
| | 3.71 | 75.54 | dd, CH |
| | 3.79 | 63.19 | dd, CH₂ |
| | 3.86 | 63.27 | dd, CH₂ |
| | 3.92 | 63.51 | dd, CH₂ |
| | 3.96 | 75.98 | dd, CH^{e} |
| | 3.98 | 75.99 | dd, CH |
| | 5.42 | 102.28 | d, CH |
| Ring B | | | |
| | 3.26 | 76.93 | dd, CH^{e} |
| | 3.29 | 76.94 | dd, CH |
| | 3.78 | 78.89 | dd, CH |
| | 3.78 | 63.43 | dd, CH2 |
| | 3.79 | 63.28 | dd, CH2 |
| | 4.66 | ~ | d^{e} |
| | 4.67 | 98.50 | d, CH |

ªMultiplicity key is as follows: d - doublet, dd - doublet of doublets; CH₂ - methylene, CH - methine protons.
^{b}α/β-Maltotriose and α/β-maltose can be free and/or as subunits within the chemical structure of amylopectin.
^{c}Homonuclear correlations observed via DQF- and TQF-COSY experiments.
^{d}Heteronuclear correlations observed via HMBC experiment.
^{e}Signals with low intensity. Human Metabolome Data Base (HMDB; http://hmdb.ca/) and literature [49] were used for confirmation of assignments.

TABLE S5
Glucose found in small intestinal samples associated
with differences found in the RM-MCCV-PLS-DA model
between the consumption of RR and rr peas¹
RR and rr Peas
Small Intestinal Samples at 60 min

| | | | P-Value | Q-Value |
|---|---|---|---|---|
| | | | for | for |
| | Chemical Shift | Association | Peas | Peas |
| Metabolite | (multiplicity) | t = 60 | t = 60 | t = 60 |
| Glucose | 3.25(dd), 3.42(m), | ↑¹ | 0.002 | 0.017 |
| | 3.49(m), 3.54(dd), | | | |
| | 3.74(m), 3.84(m), | | | |
| | 3.91(dd), 4.65(d), | | | |
| | 5.24(d) | | | |

¹Sign of association; ↑ Indicates higher excretion with RR, Multiplicity key is as follows: d—doublet, dd—doublet of doublets, m—(other) multiplet

TABLE S6
List of metabolites found in gastric samples associated
with differences found in the RM-MCCV-PLS-DA
model between the consumption of RR and rr flour
at 15′ and 30′ post ingestion¹
RR and rr Flour
Gastric Samples at 15 min

| | | | P-Value | Q-Value |
|---|---|---|---|---|
| | | | for | for |
| | Chemical Shift | Association | Flour | Flour |
| Metabolite | (multiplicity) | t = 15 | t = 15 | t = 15 |
| Amylopectin/ | 5.42(d), 3.60(dd), | ↑¹ | 0.00005 | 0.00198 |
| Maltotriose/ | 3.57(dd), 3.44(dd), | | | |
| Maltose | 3.29(dd) | | | |
| Alanine | 1.48 (d) | ↓¹ | 0.00148 | 0.02060 |
| Sucrose | 3.49(t), 3.58(dd), | ↓¹ | 0.00012 | 0.00303 |
| | 3.79 (t), 3.84(br), | | | |
| | 3.91(m), 4.06(t), | | | |
| | 4.23(d), 5.42(d) | | | |

¹Sign of association; ↑ Indicates higher excretion with RR, ↓ Indicates higher excretion with rr. Multiplicity key is as follows: d—doublet, dd—doublet of doublets, m—(other) multiple, br—(broader)

TABLE S7
List of metabolites found in small intestinal
samples associated with differences found in the
RM-MCCV-PLS-DA model between the consumption
of RR and rr flour¹
RR and rr Flour
Small Intestinal Samples at 60 min

| | | | P-Value | Q-Value |
|---|---|---|---|---|
| | | | for | for |
| | Chemical Shift | Association | Flour | Flour |
| Metabolite | (multiplicity) | t = 60 | t = 60 | t = 60 |
| Glucose | 3.25(dd), 3.42(m), | ↑¹ | 0.00009 | 0.00041 |
| | 3.49(m), 3.54(dd), | | | |
| | 3.74(m), 3.84(m), | | | |
| | 3.91(dd), 4.65(d), | | | |
| | 5.24(d) | | | |
| Sucrose | 3.49(t), 3.58(dd), | ↓¹ | 0.00033 | 0.00123 |
| | 3.79(t), 3.84(br), | | | |
| | 3.91(m), 4.06(t), | | | |
| | 4.23(d), 5.42(d) | | | |
| Gamma Amino | 2.30(t), 3.02(t) | ↓¹ | 0.00007 | 0.00035 |
| N-butyrate | | | | |
| Alanine | 1.48(d) | ↓¹ | 0.00611 | 0.01429 |

¹Sign of association; ↑ Indicates higher excretion with RR, ↓ Indicates higher excretion with rr. Multiplicity key is as follows: d—doublet, dd—doublet of doublets, m—(other) multiple, br—(broader)

TABLE S8
Demographic Characteristics of volunteers
recruited and completed Study 3¹

| Characteristics | | | | | P |
|---|---|---|---|---|---|
| (n = 10) | All Visit 1 | Visit 2 | Visit 3 | Visit 4 | Value |
| Gender (M:F) | 4:6 | | | | |
| Age (years) | 45.4 ± 4.85 | | | | |
| Weight (kg) | 70.9 ± 2.5 | 70.1 ± 2.5 | 69.4 ± 3.3 | 69.2 ± 2.8 | 0.25 |
| BMI (kg/m²) | 24.0 ± 0.9 | 24.0 ± 0.9 | 24.2 ± 1.0 | 24.0 ± 0.9 | 0.26 |
| Body Fat (%) | 28.4 ± 1.8 | 27.7 ± 2.0 | 27.2 ± 1.8 | 27.6 ± 6.7 | 0.28 |

¹Results presented as mean ± SEM

TABLE S9
Demographic Characteristics of volunteers recruited and completed Study 4¹

| | | Supplementation | | Supplementation | | |
|---|---|---|---|---|---|---|
| | | Period RR | | Period rr | | |
| | | Baseline | Baseline | Baseline | Baseline | Group x |
| Volunteers | | (Week 0) | (Week 4) | (Week 0) | (Week 4) | Time |
| (n = 25) | Screening | Visit 1 | Visit 2 | Visit 1 | Visit 2 | P Value |
| Gender (M:F) | 10:15 | — | — | — | — | — |
| Age (years) | 56.9 ± 1.3 | — | — | — | — | — |
| Weight (kg) | 71.6 ± 2.2 | 71.7 ± 2.2 | 72.4 ± 2.2 | 71.9 ± 2.2 | 72.0 ± 2.2 | 0.08 |
| BMI (kg/m²) | 24.8 ± 0.5 | 24.8 ± 0.5 | 25.0 ± 0.5 | 24.9 ± 0.5 | 25.0 ± 0.5 | 0.78 |
| Body Fat (%) | 27.1 ± 1.5 | 27.2 ± 1.4 | 27.2 ± 1.5 | 27.1 ± 1.5 | 27.0 ± 1.5 | 0.91 |

¹Results presented as mean ± SEM

TABLE S10
Macronutrient profile of RR and rr mushy peas and pea
114 hummus (per 100 g of product)₁. Each product
contained 50 g dry weight in a total weight of 115 210 g
(i.e. 100 g dry weight pea consumption/day).
Macronutrient Profile of the Test Meal

| | RR mushy | rr mushy | | |
|---|---|---|---|---|
| Macronutrients | peas | peas | RR hummus | rr hummus |
| Energy (kcal) | 61 | 50 | 187 | 187 |
| Protein (g) | 4.5 | 3.8 | 3.5 | 3.5 |
| Carbohydrates (g) | 6.7 | 5.6 | 5.6 | 5.6 |
| of which Starch | 5.1 | 4.0 | 4.3 | 4.3 |
| Sugars | 0.8 | 0.5 | 0.8 | 0.8 |
| Fat (g) | 1.1 | 0.9 | 16.3 | 16.3 |
| Saturated | 0.2 | 0.2 | 2 | 2 |
| Monounsaturated | 0.1 | 0.1 | 3.2 | 3.2 |
| Polyunsaturated | 0.5 | 0.4 | 10.2 | 10.2 |
| Trans | 0.0 | 0.0 | 0.0 | 0.0 |
| Dietary Fibre | 4 | 3 | 3 | 3 |

TABLE S11
Changes in plasma glucose, serum insulin, GLP-1, lipids and HOMA 2
biomarkers following 28 days of RR or rr pea products supplementation.

| | | | | | | | Mixed | |
|---|---|---|---|---|---|---|---|---|
| | | | | | | | Anova | |
| | | | | | | | | Group |
| | | | | | | | | x |
| | RR Line (n = 25) | | | rr Line (n = 25) | | | Time | Time |
| | | | P | | | P | P | P |
| | Pre | Post | Value | Pre | Post | Value | Value | Value |
| Laboratory | | | | | | | | |
| Outcomes | | | | | | | | |
| Mean Fasting | 5.17 ± 0.10 | 5.22 ± 0.10 | 0.45 | 5.11 ± 0.09 | 5.20 ± 0.09 | 0.21 | 0.23 | 0.86 |
| Glucose (mmol/L) | | | | | | | | |
| PPG\hat{ } (0-300 min) | 5.68 ± 0.13 | 5.63 ± 0.10 | 0.70 | 5.61 ± 0.28 | 5.80 ± 0.19 | 0.25 | 0.21 | 0.21 |
| (mmol/L)\hat{ } | | | | | | | | |
| Mean Fasting Insulin | 9.37 ± 0.82 | 9.10 ± 0.96 | 0.37 | 8.82 ± 0.82 | 9.21 ± 0.76 | 0.30 | 0.81 | 0.17 |
| (μU/mL) | | | | | | | | |
| Postprandial Insulin\hat{ } | 37.12 ± 3.37 | 34.25 ± 3.45 | 0.12 | 35.37 ± 3.09 | 34.78 ± 2.14 | 0.79 | 0.23 | 0.45 |
| (0-300 min) (μU/mL)\hat{ } | | | | | | | | |
| Mean Fasting C | 493 ± 38.35 | 497 ± 46.79 | 0.82 | 463 ± 35.14 | 498 ± 38.43 | 0.14 | 0.25 | 0.30 |
| Peptide (pmol/L)\hat{ }\hat{ } | | | | | | | | |
| GLP-1 (pmol/L)\hat{ } | 57.44 ± 1.57 | 59.95 ± 2.19 | 0.20 | 59.03 ± 1.99 | 58.74 ± 2.22 | 0.83 | 0.40 | 0.19 |
| Cholesterol (mmol/L)\hat{ } | 5.25 ± 0.9 | 4.91 ± 0.7 | 0.01 | 5.16 ± 0.8 | 5.00 ± 0.7 | 0.23 | 0.03 | 0.22 |
| Triglycerides | 0.90 ± 0.07 | 0.86 ± 0.07 | 0.23 | 0.86 ± 0.07 | 0.88 ± 0.08 | 0.70 | 0.66 | 0.37 |
| (mmol/L)\hat{ } | | | | | | | | |
| LDL Cholesterol | 3.28 ± 0.16 | 2.94 ± 0.14 | 0.004 | 3.16 ± 0.15 | 3.04 ± 0.13 | 0.22 | 0.01 | 0.05 |
| (mmol/L)\hat{ } | | | | | | | | |
| HDL Cholesterol | 1.54 ± 0.07 | 1.58 ± 0.07 | 0.31 | 1.60 ± 0.08 | 1.56 ± 0.07 | 0.27 | 0.92 | 0.11 |
| (mmol/L)\hat{ } | | | | | | | | |
| HOMA2 Beta Cell | 91.73 ± 4.56 | 88.94 ± 4.63 | 0.19 | 90.85 ± 5.64 | 90.96 ± 5.02 | 0.96 | 0.36 | 0.33 |
| Function (% B) | | | | | | | | |
| HOMA2 Insulin | 1.06 ± 0.08 | 1.04 ± 0.10 | 0.21 | 1.00 ± 0.09 | 1.05 ± 0.09 | 0.12 | 0.45 | 0.05 |
| Resistance | | | | | | | | |
| HOMA2 Insulin | 109 ± 9.02 | 113 ± 9.35 | 0.25 | 120.6 ± 12.1 | 110.68 ± 8.76 | 0.32 | 0.96 | 0.11 |
| Sensitivity (% S) | | | | | | | | |

Data are presented as mean ± SEM
\hat{ }24 volunteers and \hat{ }\hat{ }22 volunteers included in the calculations.

Supplementary TABLE 12
Nutritional information of trial 1 mixed meal test
Experimental Test Meal Nutritional Information

| Food | Unit | | CHO | Fat | Protein | Fibre |
|---|---|---|---|---|---|---|
| Products | (g) | Kcal | (g) | (g) | (g) | (g) |
| Hovis Soft | 80 | 93.0 | 17.9 | 0.7 | 3.5 | 1.0 |
| White Bread | | | | | | |
| Medium | | | | | | |
| Sliced | | | | | | |
| Heinz | 280 | 167 | 20 | 8.4 | 1.9 | 1.6 |
| Tomato | | | | | | |
| Soup | | | | | | |
| Egg | 1 (egg) | 78 | 0.6 | 5 | 6 | 0 |
| microwaved | | | | | | |
| Flora butter | 10 | 40 | 0.5 | 4.5 | 0.5 | 0 |
| Water | 500 ml | 0 | 0 | 0 | 0 | 0 |
| RR (peas and | 50 | 143 | 17.6 | <0.5 | 10.6 | 18 |
| flour) | | | | | | |
| Total (RR | | 521 | 56.6 | 19.1 | 22.5 | 20.6 |
| meal) | | | | | | |
| rr (peas and | 50 | 147 | 14 | 0.85 | 11.6 | 18.1 |
| flour) | | | | | | |
| Total (rr | | 525 | 53 | 19.4 | 23.5 | 20.9 |
| meal) | | | | | | |

SUPPLEMENTARY TABLE 13
Macronutrient profile of RR and rr peas (50 g dry weight portion)
Macronutrient profile of the test meal

| | RR peas | rr peas |
|---|---|---|
| Macronutrients | or flour | or flour |
| Energy (kcal) | 143 | 147 |
| Protein (g) | 10.6 | 11.9 |
| Carbohydrates (g) | 17.6 | 14 |
| Fat (g) | <0.5 | 0.85 |
| Dietary Fibre (g) | 18 | 18.1 |

Supplementary TABLE 14
Nutritional information of trial 3 mixed meal test
Experimental Test Meal Nutritional Information

| Food | Unit | | CHO | Fat | Protein | Fibre |
|---|---|---|---|---|---|---|
| Products | (g) | Kcal | (g) | (g) | (g) | (g) |
| Hovis Soft | 80 | 93.0 | 17.9 | 0.7 | 3.5 | 1.0 |
| White | | | | | | |
| Bread | | | | | | |
| Medium | | | | | | |
| Sliced | | | | | | |
| Heinz | 280 | 167 | 20 | 8.4 | 1.96 | 1.68 |
| Tomato | | | | | | |
| Soup | | | | | | |
| Flora butter | 10 | 40 | 0.5 | 4.5 | 0.5 | 0 |
| Water | 500 ml | 0 | 0 | 0 | 0 | 0 |
| RR (peas | 50 | 143 | 17.6 | <0.5 | 10.6 | 18 |
| and flour) | | | | | | |
| Total (RR | — | 443 | 56 | 14.1 | 16.5 | 20.6 |
| meal) | | | | | | |
| rr (peas and | 50 | 147 | 14 | 0.85 | 11.6 | 18.1 |
| flour) | | | | | | |
| Total (rr | — | 447 | 52.4 | 14.4 | 17.5 | 20.7 |
| meal) | | | | | | |

SUPPLEMENTARY TABLE 15
Macronutrient profile of RR and rr
mushy peas and pea hummus (per 100 g of product)
Macronutrient Profile of the Test Meal mushy

| | RR mushy | rr mushy | | |
|---|---|---|---|---|
| Macronutrients | peas | peas | RR hummus | rr hummus |
| Energy (kcal) | 61 | 50 | 187 | 187 |
| Protein (g) | 4.5 | 3.8 | 3.5 | 3.5 |
| Carbohydrates (g) | 6.7 | 5.6 | 5.6 | 5.6 |
| of which Starch | 5.1 | 4.0 | 4.3 | 4.3 |
| Sugars | 0.8 | 0.5 | 0.8 | 0.8 |
| Fat (g) | 1.1 | 0.9 | 16.3 | 16.3 |
| Saturated | 0.2 | 0.2 | 2 | 2 |
| Monounsaturated | 0.1 | 0.1 | 3.2 | 3.2 |
| Polyunsaturated | 0.5 | 0.4 | 10.2 | 10.2 |
| Trans | 0.0 | 0.0 | 0.0 | 0.0 |
| Dietary Fibre | 4 | 3 | 3 | 3 |

SUPPLEMENTARY TABLE 16
Total and resistant starch analysis of uncooked cotyledons from different pea varieties¹

| | | | Commercial | Commercial | Commercial |
|---|---|---|---|---|---|
| | JIC RR | JIC rr | variety Y | variety X | variety Z |
| RS g/100 g DM | 13.8 ± 0.9 | 6.5 ± 0.5 | 6.4 ± 0.4 | 7.4 ± 0.6 | 6.7 ± 0.4 |
| DS g/100 g DM | 39.1 ± 8.0 | 30.5 ± 4.0 | 33.8 ± 0.8 | 35.2 ± 1.1 | 37.6 ± 1.6 |
| TS sum g/100 g DM | 52.9 | 37.0 | 40.2 | 42.6 | 44.3 |
| | (of which | (of which | (of which | (of which | (of which |
| | 26.1% RS) | 17.6% RS) | 15.9% RS) | 17.3% RS) | 15.1% RS) |
| TS direct g/100 g DM² | 52.9 ± 0.7 | 32.4 ± 0.1 | 33.7 ± 0.4 | 35.7 ± 0.2 | 37.3 ± 0.6 |

¹Abbreviations: ‘RS’ Resistant starch; ‘DS’ Digestible starch; ‘TS’ Total starch, obtained either as the sum of RS + DS, or by direct analysis.
²Total starch measurements obtained by direct analysis method tended to be somewhat lower than the sum of DS and RS, but this does not affect the interpretation.

### REFERENCES

1. O'Keefe, J. H. and D. S. Bell, Postprandial hyperglycemia hyperlipidemia (postprandial dysmetabolism) is a cardiovascular risk factor. The American Journal of Cardiology, 2007. 100(5): p. 899-904.
2. Wolever, T. M. and C. Bolognesi, Source and amount of carbohydrate affect postprandial glucose and insulin in normal subjects. The Journal of Nutrition, 1996. 126(11): p. 2798-2806.
3. Jenkins, D., et al., Glycemic index of foods: a physiological basis for carbohydrate exchange. The American Journal of Clinical Nutrition, 1981. 34(3): p. 362-366.
4. Jenkins, D. J., et al., Glycemic index: overview of implications in health and disease. The American Journal of Clinical Nutrition, 2002. 76(1): p. 266S-273S.
5. Greenwood, D. C., et al., Glycemic index, glycemic load, carbohydrates, and Type 2 diabetes: systematic review and dose-response meta-analysis of prospective studies. Diabetes Care, 2013. 36(12): p. 4166-4171.
6. Jenkins, D. J., et al., Effect of a low-glycemic index or a high-cereal fiber diet on type 2 diabetes: A randomized trial. Jama, 2008. 300(23): p. 2742-2753.
7. Wang, S. and L. Copeland, Molecular disassembly of starch granules during gelatinization and its effect on starch digestibility: a review. Food Function, 2013. 4(11): p. 1564-1580.
8. Sievenpiper, J., et al., Effect of non-oil-seed pulses on glycaemic control: a systematic review and meta-analysis of randomised controlled experimental trials in people with and without diabetes. 2009, Springer.
9. Rayner, T., et al., Genetic variation controlling wrinkled seed phenotypes in Pisum: how lucky was Mendel? International Journal of Molecular Sciences, 2017. 18(6): p. 1205.
10. Bhattacharyya, M. K., et al., The wrinkled-seed character of pea described by Mendel is caused by a transposon-like insertion in a gene encoding starch-branching enzyme. Cell, 1990. 60(1): p. 115-122.
11. Satoh, H., et al., Starch-branching enzyme I-deficient mutation specifically affects the structure and properties of starch in rice endosperm. Plant Physiology, 2003. 133(3): p. 1111-1121.
12. Hazard, B., et al., Induced mutations in the starch branching enzyme II (SBEII) genes increase amylose and resistant starch content in durum wheat. Crop Science, 2012. 52(4): p. 1754-1766.
13. Schönhofen, A., X. Zhang, and J. Dubcovsky, Combined mutations in five wheat STARCH BRANCHING ENZYME II genes improve resistant starch but affect grain yield and bread-making quality. Journal of Cereal Science, 2017. 75: p. 165-174.
14. Sun, Y., et al., Generation of high-amylose rice through CRISPR Cas9-mediated targeted mutagenesis of starch branching enzymes. Frontiers in Plant Science, 2017. 8: p. 298.
15. Tuncel, A., et al., Cas9-mediated mutagenesis of potato starch-branching enzymes generates a range of tuber starch phenotypes. Plant Biotechnology Journal, 2019. 17(12): p. 2259-2271.
16. Wang, T. L., T. Y. Bogracheva, and C. L. Hedley, Starch: as simple as A, B, C? Journal of Experimental Botany, 1998. 49(320): p. pp. 481-502.
17. Ghoos, Y. F., et al., Measurement of gastric emptying rate of solids by means of a carbon-labeled octanoic acid breath test. Gastroenterology, 1993. 104(6): p. 1640-1647.
18. McCleary, B. V. and D. A. Monaghan, Measurement of resistant starch. Journal of AOAC International, 2002. 85(3): p. 665-675.
19. Gidley, M., et al., Molecular order and structure in enzyme-resistant retrograded starch. Carbohydrate Polymers, 1995. 28(1): p. 23-31.
20. Lopez-Rubio, A., et al., Molecular rearrangement of starch during in vitro digestion: toward a better understanding of enzyme resistant starch formation in processed starches. Biomacromolecules, 2008. 9(7): p. 1951-1958.
21. Shrestha, A. K., et al., Enzyme resistance and structural organization in extruded high amylose maize starch. Carbohydrate Polymers, 2010. 80(3): p. 699-710.
22. Skrabanja, V., et al., Influence of genotype and processing on the in vitro rate of starch hydrolysis and resistant starch formation in peas (Pisum sativum L.). Journal of Agricultural Food Chemistry, 1999. 47(5): p. 2033-2039.
23. Zhou, H. and F. Liu, Regulation, Communication, and Functional Roles of Adipose Tissue-Resident CD4+ T Cells in the Control of Metabolic Homeostasis. Frontiers in Immunology, 2018. 9.
24. Lambeth, S. M., et al., Composition, diversity and abundance of gut microbiome in prediabetes and type 2 diabetes. Journal of Diabetes, 2015. 2(3): p. 1.
25. Edwards, C. H., et al., Manipulation of starch bioaccessibility in wheat endosperm to regulate starch digestion, postprandial glycemia, insulinemia, and gut hormone responses: a randomized controlled trial in healthy ileostomy participants, 2. The American Journal of Clinical Nutrition, 2015. 102(4): p. pp. 791-800.
26. Tahir, R., et al., Study of the structure and properties of native and hydrothermally processed wild-type, lam and r variant pea starches that affect amylolysis of these starches. Biomacromolecules, 2010. 12(1): p. 123-133.
27. Edwards, C. H., et al., A comparison of the kinetics of in vitro starch digestion in smooth and wrinkled peas by porcine pancreatic alpha-amylase. Food Chemistry, 2018. 244: p. pp. 386-393.
28. Pilichiewicz, A. N., et al., Load-dependent effects of duodenal glucose on glycemia, gastrointestinal hormones, antropyloroduodenal motility, and energy intake in healthy men. American Journal of Physiology-endocrinology Metabolism, 2007. 293(3): p. E743-E753.
29. Zhang, G., Z. Ao, and B. R. Hamaker, Slow digestion property of native cereal starches. Biomacromolecules, 2006. 7(11): p. 3252-3258.
30. Donohoe, D. R., et al., The microbiome and butyrate regulate energy metabolism and autophagy in the mammalian colon. Cell Metabolism, 2011. 13(5): p. 517-526.
31. Panwar, H., et al., Probiotics as potential biotherapeutics in the management of type 2 diabetes—prospects and perspectives. Diabetes/metabolism Research Reviews, 2013. 29(2): p. 103-112.
32. Julious, S. A., Sample size of 12 per group rule of thumb for a pilot study. J Pharmaceutical Statistics: The Journal of Applied Statistics in the Pharmaceutical Industry, 2005. 4(4): p. 287-291.
33. Te Morenga, L., et al., Effect of a relatively high-protein, high-fiber diet on body composition and metabolic risk factors in overweight women. European Journal of Clinical Nutrition, 2010. 64(11): p. 1323-1331.
34. Preston, T. and D. McMillan, Rapid sample throughput for biomedical stable isotope tracer studies. Biomedical Environmental Mass Spectrometry, 1988. 16(1-12): p. 229-235.
35. Edwards, C., et al., Production of 13C labelled pea flour for use in human digestion and fermentation studies. Isotopes in Environmental Health Studies, 2002. 38(3): p. 139-147.
36. Garcia-Perez, I., et al., Identifying unknown metabolites using NMR-based metabolic profiling techniques. Nature Protocols, 2020: p. 1-30.
37. Morrison, D. J., et al., A streamlined approach to the analysis of volatile fatty acids and its application to the measurement of whole-body flux. Rapid Communications in Mass Spectrometry, 2004. 18(21): p. 2593-2600.
38 Minekus, M., et al., A standardised static in vitro digestion method suitable for food—an international consensus. Food Function, 2014. 5(6): p. 1113-1124.
39. Perez-Moral, N., et al., Ultra-high performance liquid chromatography-size exclusion chromatography (UPLC-SEC) as an efficient tool for the rapid and highly informative characterisation of biopolymers. Carbohydrate Polymers, 2018. 196: p. 422-426.
40. Flanagan, B. M., M. J. Gidley, and F. J. Warren, Rapid quantification of starch molecular order through multivariate modelling of 13 C CP MAS NMR spectra. J Chemical Communications, 2015. 51(80): p. 14856-14858.
41. EDWARDS, C. H., COCHETEL, N., SETTERFIELD, L., PEREZ-MORAL, N. & WARREN, F. J. 2019. A single-enzyme system for starch digestibility screening and its relevance to understanding and predicting the glycaemic index of food products. Food & function, 10, 4751-4760.
42. EDWARDS, C. H., RYDEN, P., PINTO, A. M., VAN DER SCHOOT, A., STOCCHI, C., PEREZ-MORAL, N., BUTTERWORTH, P. J., BAJKA, B., BERRY, S. E. & HILL, S. E. 2020. Chemical, physical and glycaemic characterisation of PulseON®: A novel legume cell-powder ingredient for use in the design of functional foods. Journal of Functional Foods, 68, 103918.

1. A method for providing a food composition, food ingredient, or food product with improved digestion properties, the method comprising a) introducing a loss of function mutation in at least one SBE1 gene of a plant from which the food composition, food ingredient, or food product is prepared; and b) processing a plant seed or root from which the food composition, food ingredient, or food product is prepared in such a way as to retain cellular structure or starch granule structure of the plant seed or root.

2. A method for providing a food composition, food ingredient, or food product with improved digestion properties, the method comprising processing a plant seed or root obtained from a plant having a loss of function mutation in at least one SBE1 gene in such a way as to retain cellular structure or starch granule structure of the plant seed or root.

3. A method for determining whether a food composition, food ingredient, or food product has improved digestion properties, the method comprising determining whether the food composition, food ingredient, or food product is prepared from a plant variety, seed or root having one or more loss-of-function mutations in at least one SBE1 gene.

4. A method for determining whether a food composition, food ingredient, or food product has improved digestion properties, the method comprising determining whether the food composition, food ingredient, or food product comprises resistant starch of the type expressed in a pea plant variety, pea or pea plant product having one or more loss-of-function mutations in at least one SBE1 gene.

5. The method of claim 1, 2 or 3 wherein the plant is a pea plant.

6. The method of claim 1, 2 or 3 wherein the plant is selected from chickpea, lentil, bean, lupin bean, soy, corn, wheat, barley, oats.

7. The method of claim 1, 2 or 3 wherein the plant is selected from parsnips; potatoes; sweet potatoes; yams; taro.

8. A method for determining whether a pea plant variety or pea or pea product derived therefrom, has improved digestion properties, the method comprising determining whether the plant or pea has one or more loss of function mutations in the SBE1 (starch branching enzyme I) gene, wherein a plant or pea with one or more mutations in the SBE1 gene has improved digestion properties.

9. A method of improving the digestion properties of a plant seed or root, the method comprising introducing one or more mutations into the SBE1 gene of the plant, wherein the one or more mutation is a loss of function or partial loss of function mutation.

10. A method of producing a plant seed or root having improved digestion properties, the method comprising introducing one or more mutations into the SBE1 gene, wherein the one or more mutation is a loss of function or partial loss of function mutation.

11. A method for identifying and selecting a plant or plant variety that has improved digestion properties, the method comprising detecting in the plant genome or germplasm at least one polymorphism in the SBE1 gene, wherein the polymorphism leads to a loss of function or a partial loss of function.

12. The method of any of claims 9-11 wherein the plant is a pea plant; or wherein the plant is selected from chickpea, lentil, bean, lupin bean, soy, corn, wheat, barley, oats; or wherein the plant is selected from parsnips; potatoes; sweet potatoes; yams; taro.

13. The method of claim 12, wherein the method further comprises introgressing the chromosomal region containing at least one polymorphism in the SBE1 gene into a second plant or plant germplasm to produce an introgressed plant or plant germplasm.

14. A method of producing a food composition or nutritional supplement, the method comprising identifying and selecting a plant or plant variety according to any of claims 9-12, and producing the food composition or nutritional supplement from the plant or part thereof, wherein preferably the part thereof is a seed.

15. The method of any preceding claim, wherein the digestion property is a reduced increase in postprandial glucose (PPG) level following consumption compared to the PPG level of a pea variety, pea or product therefrom consumed without the mutation in the SBE1 gene.

16. The method of any preceding claim, wherein the SBE1 gene encodes a polypeptide as defined in SEQ ID NO: 2 or a functional variant thereof.

17. The method of claim 16, wherein the SBE1 gene comprises a nucleic acid sequence comprising or consisting of SEQ ID NO: 1 or a functional variant thereof.

18. The method of any preceding claim, wherein the mutation is a deletion, substitution or addition of one or more nucleotides.

19. The method of any preceding claim, wherein the mutation is introduced using targeted gene editing.

20. A genetically altered plant, part thereof or plant cell, wherein the plant comprises a loss of function mutation in the SBE1 gene, wherein preferably the plant is a pea.

21. A seed or root obtained or obtainable from the genetically altered plant of claim 20.

22. A method of increasing the level of resistant starch in a pea, the method comprising introducing a loss of function mutation in the SBE1 gene.

23. A method of improving the digestion properties of a pea or pea product, the method comprising processing the seed of the pea in such a way as to enhance the relative resistant starch content.

24. A plant, part thereof or plant seed obtained or obtainable by the method of any of claims 9-12.

25. A food or feed composition derived from the plant of any of claims 9-12 or 24.

26. A method of glycaemic control, the method comprising administering a diet of the food composition of claim 25 to an individual in need thereof.
